(19) United States
(12) Patent Application Publication
(10) Pub. No.: US 2025/0049040 A1
(43) Pub. Date: Feb. 13, 2025

(54) Methods and Compositions for Growing Plants

(71) Applicant: GreenTech Ventures, Inc., Chantilly, VA (US)

(72) Inventors: Zachary Jovan POGUE, Washington. D.C., DC (US); John Kirk GRIFFIN, Plant City, FL (US)

(21) Appl. No.: 18/720,933

(22) PCT Filed: Dec. 17, 2021
(86) PCT No.: PCT/US2021/064198

§ 371 Date: Jun. 17, 2024

(51) Int. Cl. A01N 63/30; A01N 37/40; A01N 37/42; A01N 43/12; A01N 43/38; A01N 63/20; A01N 63/22; A01N 63/27; A01N 63/28; A01N 65/03; A01P 21/00

(52) U.S. Cl. CPC A01N 63/30; A01N 37/40; A01N 37/42; A01N 43/12; A01N 43/38; A01N 63/20; A01N 63/22; A01N 63/27; A01N 63/28; A01N 65/03; A01P 21/00

(57) ABSTRACT

Disclosed herein are methods of growing a plant, comprising applying to the plant and/or growth medium (a)an effective amount of a composition comprising one or more mycorrhizal fungi and/or one or more mycorrhiza helper bacteria (MHB)[A1] and (b) an effective amount of a composition comprising one or more gibberellins, one or more auxins, salicylic acid, and/or one or more jasmonates. Also disclosed herein are mixtures and uses of the compositions. The compositions can improve crop yield of plants and boost microbial activity in the soil.

### FIELD

Disclosed herein are methods of growing a plant, comprising applying to the plant and/or growth medium for the plant (a) an effective amount of a composition comprising one or more mycorrhizal fungi and/or one or more mycorrhiza helper bacteria (MHB) and (b) an effective amount of a composition comprising one or more gibberellins, one or more auxins, salicylic acid, and/or one or more jasmonates. Also disclosed herein are mixtures and uses of the compositions. The compositions can improve crop yield of plants and boost microbial activity in the soil.

### BACKGROUND

Supplementing or amending the soil in which crops are grown is common practice in agriculture, leading to an improvement in the health of the soil, increase in the population and diversity of beneficial microorganisms in the soil microbiome, the health of plants, improved root system, enhanced growth, and thus an increased yield and improved crop quality and shelf life for the grower. The application of fertilizers is well known and understood, with most being based around the key nutrients of nitrogen (N), phosphorous (P), and potassium (K), as well as micronutrients. Such fertilizers are well known in the art.

Leaving the traditional fertilizers to one side, there is a burgeoning class of other types of soil supplements (or amendments) based around other factors, which are used to improve plant and soil health, and are becoming increasingly used in commercial agriculture. These typically work by improving the condition of the soil, improving the microbial ecosystem in the soil, and allowing key nutrients to be better assimilated and absorbed by the plant. Several common amendments that are available are derived from the biomass of other organisms, such as manure, bracken, seaweed, and peat moss. Another example, soil conditioners alter the texture and porosity of the soil in order to encourage root growth, allow increased aeration and improved drainage of the soil. Another example is the use of mycorrhizal fungi, such as, but not limited to, arbuscular mycorrhizal fungi (AMF or AM), also known as vesicular arbuscular mycorrhizae (VAM) or endomycorrhizae, and ectomycorrhizae fungi (EMF or EM), ericoid mycorrhizal fungi (commonly grouped with arbutoid and monotropoid), orchid mycorrhizal fungi (OMF), as a growing media and root inoculant.

There continues to be a need for improvement in crop yield and quality.

### SUMMARY OF THE INVENTION

Disclosed herein are methods of growing a plant, comprising applying to the plant and/or growth medium for the plant (a) an effective amount of a composition comprising one or more mycorrhizal fungi and/or one or more mycorrhiza helper bacteria (MHB) and (b) an effective amount of a composition comprising one or more gibberellins, one or more auxins, salicylic acid, and/or one or more jasmonates. The applying of (a) and (b) can occur concurrently or on the same day. The applying of (b) can occur 1 to 10 days after the applying of (a). The methods can further comprise applying the composition of (b) every 7 to 60 days after the applying of (a). The MHB can be one or more species in the genera Bacillus, Pseudomonas, and/or Streptomyces. The PGPR can be Pseudomonas putida, Azospirillum fluorescens, Azospirillum lipoferum, and/or one or more species in the genera Azotobacter, Klebsiella, Enterobacter, Alcaligenes, Arthrobacter, Burkholderia, Bacillus, Serratia, Allorhizobium, Azorhizobium, Bradyrhizobium, and/or Rhizobium.

Disclosed herein are method of growing a plant, comprising (a) providing an algae culture and air to an algae culture broth composition producing system, the system comprising: a sterilizer; an automatic carbon dioxide supply device to promote photosynthesis; an at least partially or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and a high-efficiency harvesting device using hollow fiber membranes; (b) harvesting the composition; (c) applying an effective amount of the composition to the plant and/or the growth medium for the plant; and (d) apply an effective amount of a composition comprising one or more mycorrhizal fungi and/or one or more mycorrhiza helper bacteria (MHB).

Disclosed herein are mixtures comprising (a) an effective amount of a composition comprising one or more mycorrhizal fungi and/or one or more mycorrhiza helper bacteria (MHB) and (b) a composition comprising one or more gibberellins, one or more auxins, salicylic acid, and/or one or more jasmonates.

Disclosed herein are uses of one or more compositions for growing a plant, comprising: (a) an effective amount of a composition comprising one or more mycorrhizal fungi and/or one or more mycorrhiza helper bacteria (MHB) and (b) an effective amount of a composition one or more gibberellins, one or more auxins, salicylic acid, and/or one or more jasmonates.

In some embodiments, the methods or uses can further comprise applying an effective amount of a nitrogen/phosphorus/potassium (NPK) fertilizer, with or without macro- and/or micro-nutrients, to the plant and/or growth medium for the plant. In some embodiments, the mixtures can further comprise an NPK fertilizer.

In some embodiments, prior to an optional dilution of the composition in (b): the one or more gibberellins are at a concentration of 0.01 to 1 ng/ml; the one or more auxins are at a concentration of 0.001 to 1 ng/ml; the salicylic acid is at a concentration of 0.1 to 1 ng/ml; and/or the one or more jasmonates are at a concentration of 0.001 to 1 ng/ml. In other embodiments, the one or more gibberellins are at a concentration of 0.01 to 1 ng/ml; the one or more auxins are at a concentration of 0.001 to 1 ng/ml; the salicylic acid is at a concentration of 0.1 to 1 ng/ml; and/or the one or more jasmonates are at a concentration of 0.001 to 1 ng/ml; and further comprising diluting the composition to 25:1 and 150:1 prior to applying the effective amount.

In some embodiments, the composition in (b) is derived from algae, macroalgae, and/or microalgae. In some embodiments, the microalgae comprise Chlorella, Spirulina, Nannochloropsis, and/or Scenedesmus or combinations thereof. In some embodiments, the one or more gibberellins comprise GA1, GA3, GA4, GA5, GA6, GA7, or combinations thereof. In some embodiments, the one or more auxins comprise Me-IAA, IAA-ALA, IAA-ASP, IBA, or combinations thereof. In some embodiments, the one or more jasmonates comprise jasmonic acid.

In some embodiments, the composition in (b) further comprises cultured and enhanced sterilized water, optionally at 80% to 99.5% w/w. The composition in (b) comprises biomass, ethylene, abscisic acid, brassinolides, or combinations thereof. The composition in (b) does not comprise biomass, ethylene, abscisic acid, brassinolides, or combinations thereof.

In some embodiments, the plant is from any of the following mycorrhizal host plant families: Actinidiaceae, Adoxaceae, Allaceae, Anacardiaceae, Apiaceae (Umbelliferae), Arecaceae, Asteraceae, Bromeliaceae, Cactaceae, Caesalpinioideae, Cannabaceae, Capparaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Rosaceae, Rutaceae, Sapindaceae, Saxifragaceae, Solanaceae, and/or Vitaceae.

The composition in (b) can be derived from algae, macroalgae, and/or microalgae. In some embodiments, the microalgae can comprise Chlorella, Spirulina, Nannochloropsis and/or Scenedesmus or any combination thereof. In some embodiments, the effective amount of the composition in (a) or (b) is applied to the one or more seeds, the soil, growth medium, and/or a cultivated area, and wherein the effective amount is 1 to 5 liters per acre.

The methods can further comprise diluting with an aqueous solution the composition in (b) in a ratio of 50:1 and 300:1, respectively, and performing one or more additional applications of the effective amount of the composition.

In some embodiments, the applying comprises injection, in-furrow, drip irrigation, fertigation, foliar application, seed placement, seed treatment, or combinations thereof.

### BRIEF DESCRIPTION OF THE FIGURES

FIG. 1 shows a process flow diagram for producing a composition disclosed herein.

FIG. 2 shows an overview of the process for producing a composition disclosed herein.

### DETAILED DESCRIPTION OF THE INVENTION

It has been determined that compositions comprising one or more gibberellins and/or one or more auxins and/or salicylic acid and/or one or more jasmonates can be used to improve the efficiency of mycorrhizal fungi, mycorrhizal soil and/or root inoculation, and/or MHB products to provide a variety of benefits, including, but not limited to, plant growth and yield.

Disclosed herein are methods of growing a plant, comprising applying to the plant and/or growth medium for the plant (a) an effective amount of a composition comprising one or more mycorrhizal fungi and/or one or more mycorrhiza helper bacteria (MHB) and (b) an effective amount of a composition comprising one or more gibberellins, one or more auxins, salicylic acid, and/or one or more jasmonates. In some embodiments, disclosed herein are methods of growing a plant, comprising applying to the plant and/or growth medium for the plant (a) an effective amount of a composition comprising one or more mycorrhiza helper bacteria (MHB) and (b) an effective amount of a composition comprising one or more gibberellins, one or more auxins, salicylic acid, and/or one or more jasmonates. The applying of (a) and (b) can occur concurrently or on the same day. In some embodiments, the composition in (a) and the composition in (b) are combined before concurrent application. The applying of the composition in (b) can occur 1 to 10 days after the applying of the composition in (a). The methods can further comprise applying the composition of (b) every 7 to 60 days after the applying of (a). The MHB can be one or more species in the genera Bacillus, Pseudomonas, and/or Streptomyces. The PGPR can be Pseudomonas putida, Azospirillum fluorescens, Azospirillum lipoferum, and/or one or more species in the genera Azotobacter, Klebsiella, Enterobacter, Alcaligenes, Arthrobacter, Burkholderia, Bacillus, Serratia, Allorhizobium, Azorhizobium, Bradyrhizobium, and/or Rhizobium.

Disclosed herein are method of growing a plant, comprising (a) providing an algae culture and air to an algae culture broth composition producing system, the system comprising: a sterilizer; an automatic carbon dioxide supply device to promote photosynthesis; an at least partially or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and a high-efficiency harvesting device using hollow fiber membranes; (b) harvesting the composition; (c) applying an effective amount of the composition to the plant and/or the growth medium; and (d) apply an effective amount of a composition comprising one or more mycorrhizal fungi and/or one or more mycorrhiza helper bacteria (MHB). In some embodiments, the composition in (d) comprises one or more mycorrhiza helper bacteria (MHB). In some embodiments, the composition can be harvested by separating the composition from biomass of the algae culture using a harvesting device, optionally dehydrating the composition for storage and transport, and optionally diluting it prior to use.

Disclosed herein are mixtures comprising (a) an effective amount of a composition comprising one or more mycorrhizal fungi and/or one or more mycorrhiza helper bacteria (MHB) and (b) a composition comprising one or more gibberellins, one or more auxins, salicylic acid, and/or one or more jasmonates. In some embodiments, disclosed herein are mixtures comprising (a) an effective amount of a composition comprising one or more mycorrhiza helper bacteria (MHB) and (b) a composition comprising one or more gibberellins, one or more auxins, salicylic acid, and/or one or more jasmonates.

Disclosed herein are uses of one or more compositions for growing a plant, comprising: (a) an effective amount of a composition comprising one or more mycorrhizal fungi and/or one or more mycorrhiza helper bacteria (MHB) and (b) an effective amount of a composition one or more gibberellins, one or more auxins, salicylic acid, and/or one or more jasmonates. In some embodiments, disclosed herein are uses of one or more compositions for growing a plant, comprising: (a) an effective amount of a composition comprising one or more mycorrhiza helper bacteria (MHB) and (b) an effective amount of a composition one or more gibberellins, one or more auxins, salicylic acid, and/or one or more jasmonates.

The methods disclosed herein can increase, improve, or enhance crop yield, marketable weight, mycorrhizal root colonization, mycorrhizal root spore uptake, soil microbial population and diversity in a plant growth medium (such as soil) treated with commercial and non-commercial mycorrhizal products, and or mycorrhizal products with mycorrhizal helper bacterium (MHB), including plant growth-promoting rhizobacteria (PGPR) and/or plant growth-promoting bacteria (PGPB), to amplify those beneficial mechanisms.

In some embodiments, the methods and mixtures disclosed herein can improve crop yield by 10%-25%, 25%-50%, 50%-75%, 75%-100%, 100%-125%, 125%-150%, 150%-175%, 175%-200%, 200%-300%, or any percent or ranges of percent derived therefrom compared to the use of a composition comprising one or more mycorrhizal fungi and/or one or more mycorrhiza helper bacteria (MHB) without a composition one or more gibberellins, one or more auxins, salicylic acid, and/or one or more jasmonates.

“Mycorrhizal fungi” are soil microorganisms able to form mutualistic symbiosis with most terrestrial plants and their multiple benefits to plant growth and soil health are well known. The multiple benefits of mycorrhizal fungi include improved nutrient acquisition and greater biomass leading to increased surface area to allow for more efficient absorption of soil nutrients and water.

Mycorrhizal fungi taxonomy is generally known as follows:
Acaulospora: Eukaryota; Fungi/Metazoa group; Fungi; Glomeromycota; Glomeromycetes; Diversisporales; Acaulosporaceae; Acaulospora; A. laevis.
Entrophospora: Eukaryota; Fungi/Metazoa group; Fungi; Glomeromycota; Glomeromycetes; Diversisporales; Acaulosporaceae; Entrophospora; E. infrequens.
Funneliformis: Eukaryota; Fungi/Metazoa group; Fungi; Glomeromycota; Glomeromycetes; Glomerales; Glomeraceae; F. mosseae.
Gigaspora: Eukaryota; Fungi/Metazoa group; Fungi; Glomeromycota; Glomeromycetes; Diversisporales; Gigasporaceae; G. margarita, Cetraspora, Dentiscutata, Quatunica, Racocetra, Scutellospora.
Glomus: Eukaryota; Fungi/Metazoa group; Fungi; Glomermycota; Glomermycetes; Glomerales; Glomeraceae; G. monosporus (some members of the genus were originally described as Sclerocystis species, but this genus has been entirely transferred to Glomus).
Pisolithus: Eukaryota; Fungi/Metazoa group; Fungi; Basidiomycota; Agaricomycetes; Boletales; Sclerodermataceae; Pisolithus; P. tinctoris.
Rhizophagus: Eukaryota; Fungi/Metazoa group; Fungi; Glomermycota; Glomermycetes; Glomerales; Glomeraceae; R irregularis.
Rhizopogon: Eukaryota; Fungi/Metazoa group; Fungi; Basidiomycota; Agaricomycetes; Boletales; Rhizopogonaceae; Rhizopogon; R. amylopogon.
Scleroderma: Eukaryota; Fungi/Metazoa group; Fungi; Basidiomycota; Agaricomycetes; Boletales; Sclerodermataceae; Scleroderma; S. citrinum.
Septoglomus: Eukaryota; Fungi/Metazoa group; Fungi; Glomermycota; Glomermycetes; Glomerales; Glomeraceae; S. deserticola.

Mycorrhizal fungi are soil supplements (or amendments) that are used to improve plant and soil health. These typically work by improving the condition of the soil, improving the microbial ecosystem in the soil, and allowing key nutrients to be better assimilated and absorbed by the plant. Mycorrhizal fungi include but are not limited to (1) arbuscular mycorrhizal fungi (AMF or AM), also known as vesicular arbuscular mycorrhizae (VAM) or endomycorrhizae, (2) ectomycorrhizae fungi (EMF or EM), (3) ericoid mycorrhizal fungi (commonly grouped with arbutoid and monotropoid), and (4) orchid mycorrhizal fungi (OMF), that can be introduced into a growing media as a soil and/or root inoculant, i.e., mycorrhiza soil and/or root inoculant.

“Arbuscular mycorrhizal fungi” (AMF or AM), also known as “vesicular arbuscular mycorrhizae” (VAM) or “endomycorrhizae,” can be characterized by their formation of unique specialized structures, such as arbuscules and vesicles, which both play a role in helping plants capture vital macro- and macro-nutrients in the soil that are typically unavailable in their unconverted, insoluble form or beyond the reach of the roots given they reside outside the rhizosphere, the soil that surrounds and is influenced by the roots of a plant. This highly evolved mutualistic symbiosis found between AMF and plants can be found in over 80% of vascular plant families in existence today and is said to have played a crucial role in the initial colonization of land by plants and in the evolution of vascular plants. AMF include but are not limited to: Glomus aggregatum, Glomus brasilianum, Glomus clarum, Glomus deserticola, Glomus etunicatum, Glomus intraradices (now known as Rhizophagus intraradices or Rhizophagus irregularis), Glomus margarita, Glomus monosporus and Glomus mosseae). Nearly 90% of plant species including flowering plants, bryophytes, and ferns can develop interdependent connections with AMF (Reference 4).

Upon plant need, the AMF is permitted entry into the cell membrane and is able to stretch into the root cortex, resulting in a newly developed biological bridges between the root and growing media, typically soil. Additionally, new specialized structures are formed by the AMF to facilitate this relationship, namely arbuscules and vesicles, serving as sites of nutrient exchange within the cell and storage sites, respectfully. Plant processes are improved due to this symbiosis given the better access to and uptake of diffusion-limited nutrients and water in the growing media, which the AMF continue to support in exchange for the photosynthetic carbons from the plant. AMF can help plants allocate a range of mineral macro- and micro-nutrients, from immobile elements like phosphorus (P), zinc (Zn), and copper (Cu) to more mobile ions like sulfur (S), calcium (Ca), sodium (Na), potassium (K), iron (Fe), magnesium (Mg), manganese (Mn), chlorine (Cl), bromine (Br), and nitrogen (N). Enhanced plant performance, as evidenced by improved plant growth and/or increased yield, can be attributed to the increase in efficiency of mineral uptake by the roots in the growing media where such elements may be deficient or otherwise less available and/or the moderation of certain nutrient uptake in growing media containing high levels of minerals thus preventing plant toxicity, largely due to the AMF symbiosis.

The beneficial plant responses can be attributed to these AMF mechanisms: 1) increased physical exploration of the soil due to increases of surface area (up to 50×) by AMF extrametrical hyphae within a growing media volume for nutrient absorption that extend beyond the root zone (4-20 cm+) that has already been depleted of water and available nutrients, 2) increased solubilization of generally insoluble nutrients due to unique AMF enzymatic capabilities (e.g., phosphatase converts phosphate into soluble forms for more phosphorus uptake) and root molecular changes (e.g., increase in acid phosphatase in root increasing capability of plant to convert insoluble organic P into bioavailable forms of P, and conversion of insoluble N to bioavailable forms due to increase decomposition with subsequent capture of inorganic N from complex organic materials and enhanced degradation or N mineralization of organic residues) leading to increased nutrient availability, 3) increased rate of nutrient uptake due to AMF hyphae having a higher affinity for mineral ions combined with a lower threshold concentration for with finer absorption (2 μm compared to 10-20 μm of root hair and 100-500 μm fine-root diameters) resulting in approximately 10×-100× more efficiency than that of root hairs and fine roots, respectively, 4) improved root storage and accessibility of absorbed minerals and lipids by the plant during times of limited supply due to AMF specialized vesicle structures, 5) improved buffering against nutrient lockout conditions and mitigation of uptake of excess salts and toxic minerals due to AMF protecting plants from saline conditions and heavy metal uptake through balancing of toxic Na+ and Cl− uptake for that of other ions (e.g., K+, Ca2+, and Mn2+) and regulated prevention of the dangerous accumulation of Cu, Zn, Arsenic (As), A1 that can cause slow growth, chlorosis, root browning, and plant death by the AMF's ability to sequester heavy metals in the fungal tissue and improved phosphorous nutrition of the plant, and 6) reduction of crop inputs due to the increased hyphal mass, improved plant root connection to the growing media, and making more bioavailable typically unavailable nutrients (Reference 2).

In addition to AMF, EMF are also important inoculants as they are synergistic with another large grouping of plants with another larger grouping of plants (6,000+) and contribute to both nutrient and water uptake, particularly those nutrients presenting low mobility in the soil, such as phosphorus (Reference 14). The hyphae of the ectomycorrhizal fungi grow out beyond the root zone and, their colonized roots are generally more branched than the uncolonized roots (Reference 15). Other benefits of EMF can include its ability to augment hydraulic conductivity inside the plant, improve the resistance of the plant to drought and soil-borne pathogens, and lastly can facilitate the improvement of soil aggregation and structure (References 16 and 17). Through these different mechanisms, EMF can promote plant growth and productivity even in low fertility, challenged, or disturbed soils (References 16 and 18). In contrast to AMF inoculants, which are presented as different types of commercial products and sold by several companies, only relatively few EMF, ericoid, and/or OMF inoculants have been commercialized.

“Ectomycorrhizae fungi” (EMF or EM) can be characterized by their hyphae forming a mantle around the root and also growing into the spaces between root cells, but they do not penetrate the root cells unlike AMF. EMF hyphae form a net-like covering, called a Hartig net, around the cells, which extends into the root, penetrating between epidermal and cortical cells. This network is a site of nutrient exchange between the fungus and the host plant. EMF associations are formed mostly on the fine root tips of the host plant and are more abundant in topsoil layers that contain humus. There are thousands of EMF species that exist and they are typically formed between the roots of around 10% of plant families and some of these fungi are symbiotic with only one particular genus of plant, while others form mycorrhizas associations with many different types of plants (Reference 24). EMF include but are not limited to: Pisolithus tinctoris, Rhizopogon amylopogon, Rhizopogon fulvigleba, Rhizopogon luteolus, Rhizopogon villosulus, Scleroderma cepa, and Scleroderma citrinum).

“Ericoid mycorrhizal fungi” can form a symbiotic relationship between fungi and the roots of several crop plants and ornamental species from the order Ericales (e.g., tea, blueberry, cranberries, azalea, and Rhododendron), which includes Ericaceous plants that commonly grow in low-nutrient, depauperate soils, where the ericoid mycorrhizal fungi enable their host plants to obtain these scarce nutrients (Reference 22).

“Orchid mycorrhizal fungi” (OMF) can provide the nutrients (especially carbohydrates), needed for orchids of plant family Orchidaceae (20,000+ species identified) to grow, and in many cases, orchid seeds will not germinate unless they have been infected by an appropriate fungus. OMF infection of an orchid seed can occur after the embryo takes up water and swells, rupturing the seed coat, where the embryo then emerges with a few root hairs produced, which the OMF hyphae rapidly colonize. As hyphae penetrate a cell of the embryo, the plasma membrane of the orchid cell invaginates, and the hypha becomes surrounded by a thin layer of cytoplasm. An orchid embryo contains only a few hundred cells and the fungi spread quickly from cell to cell. A successful fungal infection can result in the germination of an orchid seed. OMF may be the sole source of nutrition during the first years of life, but most orchid species develop chlorophyll in their adult stage and therefore become less dependent on the OMF. However, most still have mycorrhizal roots and gain nitrogen and phosphorus from the fungus (Reference 19).

“Mycorrhiza helper bacteria” (MHB) (e.g., Bacillus coagulans) can serve a role in increasing symbiosis development and improving mycorrhizae efficiency. Given the rich diversity of root exudates and plant cell debris that attract diverse and unique patterns of microbial colonization, the rhizomicrobiome is of great importance to agriculture. Within the rhizomicrobiome are many microbes that play key roles in nutrient acquisition and assimilation, improved soil texture, secreting, and modulating extracellular molecules such as hormones, secondary metabolites, antibiotics, and various signal compounds, all leading to enhancement of plant growth. MHB can include plant growth-promoting bacteria (PGPB) or plant growth-promoting rhizobacteria (PGPR), and can be combined with mycorrhizal fungi to amplify those aforementioned beneficial mechanisms. There are many species of MHB (e.g., Bacillus coagulans) that can serve a role in increasing symbiosis development and improving mycorrhizae efficiency. Given the rich diversity of root exudates and plant cell debris that attract diverse and unique patterns of microbial colonization, the rhizomicrobiome is of great importance to agriculture. Within the rhizomicrobiome, there are many microbes that play key roles in nutrient acquisition and assimilation, improved soil texture, secreting, and modulating extracellular molecules such as hormones, secondary metabolites, antibiotics, and various signal compounds, all leading to enhancement of plant growth. PGPR include multiple genera, such as: Pseudomonas, Azospirillum, Azotobacter, Klebsiella, Enterobacter, Alcaligenes, Arthrobacter, Burkholderia, Bacillus, and Serratia, which enhance plant growth and yield production (References 8 & 9).

“Mycorrhizal soil inoculant” and/or “mycorrhizal root inoculant” (also generally, “mycorrhizal inoculant”) means products containing mycorrhizal fungi.

Mycorrhizal soil and/or root inoculant or inoculation benefits can be enhanced through the presence of MHB. Certain bacteria, such as Bacillus coagulans in combination with a variety of AMF inoculum (such as Glomus fasciculatum, Glomus mossae, and Glomus caledonium) can improve a variety of plant responses (plant height, number of shoots, number of leaves/plant, fresh leaf yield, leaf phosphorous concentration, stem girth, number of flowers/plant, number of fruits/plant, fruits harvested, fruit weight) and improve overall AMF colonization, even when there has been a forced reduction of 50% in the recommended phosphorous (P) fertilizer for established field crops. (Reference 1).

Techniques for determining the effects of mycorrhizal soil or root inoculant and/or MHB on plant and soil health include various plant phenotype counts and observations, various mycorrhizal root and spore count evaluations, microbial population tests, Normalized Difference Red Edge (NDRE)/Normalized Difference Vegetation Index (NDVI) imaging evaluation, and final harvest counts. The optimal values for plants are derivable for plants using routine empirical scientific methods and efficacy of adding mycorrhizal soil and/or root inoculant and/or MHB treatments can be determined by comparing Control versus Treated plants.

Mycorrhizal soil and/or root inoculants and/or MHB mycelium and/or spores (e.g., Glomus aggregatum, Glomus brasilianum, Glomus clarum, Glomus deserticola, Glomus etunicatum, Glomus intraradices (now known as Rhizophagus intraradices or Rhizophagus irregularis), Glomus margarita, Glomus monosporus, and Glomus mosseae); propagules of ectomycorrhizae mycelium and/or spores (e.g., Pisolithus tinctoris, Rhizopogon amylopogon, Rhizopogon fulvigleba, Rhizopogon luteolus, Rhizopogon villosulus, Scleroderma cepa, Scleroderma citrinum); MHB (e.g., Acinetobacter, Acinetobacter calcoaceticus, Arthrobacter globiformis, Aspergillus, Azobacter, Azotobacter vinelandii, Bacillus coagulans, Bacillus Cereus, Bacillus licheniformis, Bacillus Megatarium, Bacillus pumilus, Bacillus subtilis, Bacillus Thuringiensis, Basillus, Lactobacillus, Pseudomonas, Rhizobium Meliloti, Rhizobium Phaseoli, Streptomyces griseus, Subtillus, Trichoderma afroharzianum, Trichoderma atroviride, and Trichoderma virens); mycorrhizal feeder substrate (i.e., naturally occurring paramagnetic & diamagnetic ground rock mineral powders, high nutrient content of protein and carbohydrates derived from plants and herbs plus geologically concentrated humus humates); humic acid; and macroalgae extracts (e.g., kelp (soluble Ascophyllum nodosum seaweed)).

MHB can include “plant growth-promoting bacteria” (PGPB), which can also include “plant growth-promoting rhizobacteria” (PGPR), including but not limited to the following bacterial genera: Pseudomonas, Azospirillum, Azotobacter, Klebsiella, Enterobacter, Alcaligenes, Arthrobacter, Burkholderia, Bacillus, and Serratia, which enhance plant growth and yield production (References 8, 9, and 23).

In some embodiments, the methods or uses can further comprise applying an effective amount of a nitrogen/phosphorus/potassium (NPK) fertilizer, with or without macro- and/or micro-nutrients, to the plant and/or growth medium for the plant. In some embodiments, the mixtures can further comprise an NPK fertilizer. NPK fertilizers can include but are not limited to phosphorus (P), zinc (Zn), copper (Cu), sulfur (S), calcium (Ca), sodium (Na), potassium (K), iron (Fe), magnesium (Mg), manganese (Mn), chlorine (Cl), bromine (Br), and/or nitrogen (N).

As used herein, the terms “increasing,” “enhancing,” and “improving” can be used interchangeably and can be compared to a control without the compositions or performance of the methods disclosed herein.

As used herein, the term “uptake” means absorption, such as by a plant. As used herein, a “plant” includes all its parts, including but not limited to leaves, stems, fruits, flowers, roots, and root system.

As used herein, a plant “growing in soil treated with mycorrhizal fungi, mycorrhizal soil and/or root inoculant, and/or MHB” means that the plant, parts of the plant, and/or soil in which the plant is growing has been treated with the mycorrhizal soil or root inoculant and/or MHB product.

As used herein, unless specified, “growth medium,” growth media,” “growing media,” “growing medium,” or “soil” includes any growing media or soil suitable for growing plants, including soils or non-soil media that are particularly suitable for growing a particular plant indoors and/or outdoors.

As used herein, “apply,” applying,”, or “application” means the plant or growth media for the plant is treated with one or compositions disclosed herein, such as by but not limited to injection, in-furrow, drip irrigation, band application, fertigation, foliar application, seed placement and/or seed treatment, or any combinations thereof.

In some embodiments, the applying in (a) and (b) can occur concurrently or on the same day. In some embodiments, the applying in (b) occurs 1 to 9 days after the applying in (a). In some embodiments, the applying in (b) occurs 1, 2, 3, 4, 5, 6, 7, 8, 9, or 10 days after the applying in (a). As used herein, the term “within” 10 days includes the 10th day, e.g., if mycorrhizal soil and/or root inoculant and/or MHB and/or PGPR/PGPB is applied on Day1, and a composition disclosed herein is applied “within” 10 days, it would include applying up to Day 11. The methods disclosed herein refers to each time an mycorrhizal soil and/or root inoculant and/or MHB and/or PGPR/PGPB is applied, such that each time a plant is applied with an mycorrhizal soil and/or root inoculant and/or MHB and/or PGPR/PGPB, the methods disclosed herein can be utilized. For example, if a plant is applied with a mycorrhizal fungi inoculant and/or MHB and/or PGPR/PGPB multiple times during its lifecycle, the methods disclosed herein can be utilized after each application of the mycorrhizal fungi inoculant and/or MHB and/or PGPR/PGPB.

In other embodiments, the methods can further comprise applying the composition in (a) every 1 to 60 days after the applying in (b). In some embodiments, the methods can further comprise applying the composition in (a) once every 1 to 30 days, once per month, once every 1 to 14 days, once every 7 days, or once every 5 days, or once every number of days therein.

In some embodiments, the effective amount is applied to the one or more seeds, soil, growing media, and/or the cultivated area, and wherein the effective amount is 1 to 5 liters per acre.

Further disclosed herein are methods of producing compositions that increase the efficiency of mycorrhizal fungi inoculation and/or MHB and/or PGPR/PGPB products and plant growth and yield, described herein and further comprising applying the composition to a plant by the methods described herein.

Also disclosed herein are mixtures comprising a mycorrhizal fungi, mycorrhizal inoculant, and/or MHB, such as PGPR or PGPB and a composition comprising one or more gibberellins, one or more auxins, salicylic acid, and/or one or more jasmonates. In some embodiments, prior to an optional dilution, the composition comprises: the one or more gibberellins are at a concentration of 0.01 to 1 ng/ml; the one or more auxins are at a concentration of 0.001 to 1 ng/ml; the salicylic acid is at a concentration of 0.1 to 1 ng/ml; and/or the one or more jasmonates are at a concentration of 0.001 to 1 ng/ml.

Also disclosed herein are the use of a composition to enhance the efficiency of mycorrhizal fungi and/or MHB products on plant growth and yield, the composition comprising: one or more gibberellins; one or more auxins; salicylic acid; and/or one or more jasmonates.

In some embodiments, prior to an optional dilution of the applying in (b), the composition comprises the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01-1 ng/ml, the one or more auxins are at a concentration of 0.001 to 0.5 ng/ml, the salicylic acid is at a concentration of 0.5-1 ng/ml, and/or the one or more jasmonates are at a concentration of 0.001 to 0.1 ng/ml; or the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml. In some embodiments, the mixtures comprising a mycorrhizal fungi, mycorrhizal soil and/or root inoculant, MHB, and/or PGPR/PGPB and a composition disclosed herein can be diluted before applying to the seed, soil, or plants.

Gibberellins are a large family of plant hormones involved in various aspects of plant growth. There are approximately 126 known gibberellins which have been identified in plants, fungi, and bacteria. Of these, there is a known subset of active gibberellins which are thought to be key in regulating biological processes in plants. In some embodiments, the gibberellins comprise GA1, GA3, GA4, GA5, GA6, GA7, GA8, GA9, GA13, GA14, GA15, GA19, GA20, GA24, GA29, GA44, GA51, and/or GA53, or any combination thereof. In some embodiments, the gibberellins comprise GA1, GA3, GA4, GA6, GA7, GA8, GA9, GA13, GA14, GA15, GA19, GA20, GA24, GA29, and/or GA44. In some embodiments, a composition comprising one or more gibberellins comprising any one of GA1, GA3, GA4, GA5, GA6 and/or GA7, or any combination thereof can produce an efficiency of mycorrhizal fungi, mycorrhizal soil and/or root inoculation, MHB, and/or PGPR/PGPB on plant growth and yield.

In some embodiments, the composition comprises GA1, GA3, GA4, GA6, and/or GA7 or any combination thereof. Surprisingly, applicants have found that a low concentration of gibberellins maintains this effect. While higher concentrations of gibberellins can be beneficial, it has been surprisingly found that even very low concentrations of gibberellins produce the advantageous properties of the compositions disclosed herein. In some embodiments, the gibberellins are in the composition at a concentration of 0.01 to 1 ng/ml, 0.05 to 1 ng/ml, or 0.01 to 0.8 ng/ml. In some embodiments, these concentrations are prior to a dilution into water or aqueous media before use. The gibberellins are known to stimulate material mobilization during germination, stimulate pollen tube growth and promote elongation of stems.

Auxins are a different family of plant hormones which control the growth of plants by promoting mitosis and elongation of plant cells. In some embodiments, the auxins in the present composition comprise Me-IAA, IAA, IBA, IAA-ASP, IAA-GLU, and/or IAA-ALA, or any combination thereof. In some embodiments, the auxins in the present composition comprise IAA, IBA, IAA-ASP, and IAA-ALA. While higher concentrations of auxins can be beneficial, it has been surprisingly found that even very low concentrations of auxins produce the advantageous properties of the compositions disclosed herein. In some embodiments, the auxins are in the composition at a concentration of 0.001 to 1 ng/ml, 0.001 to 0.5 ng/ml, or 0.001 to 0.4 ng/ml. In some embodiments, these concentrations are prior to a dilution into water or aqueous media before use.

Salicylic acid is known to regulate genes involved in plant defense mechanisms, enhancing germination, flowering, and ripening. While higher concentrations of salicylic acid can be beneficial, it has been surprisingly found that even very low concentrations of salicylic acid produce the advantageous properties of the compositions disclosed herein. In some embodiments, salicylic acid is present in the composition as a concentration of 0.1 to 1 ng/ml, 0.5 to 1 ng/ml, 0.2 to 0.8 ng/ml, or 0.5 to 0.75 ng/ml. In some embodiments, these concentrations are prior to a dilution into water or aqueous media before use.

Jasmonates are known to play a role in germination and the response to environmental stresses, including but not limited to defense against insects and key necrotrophic pathogens. In some embodiments, the jasmonates of the composition comprise jasmonic acid, 12-oxophytodienoic acid (OPDA), and/or jasmonoyl-isoleucine (JA-Ile) or any combination thereof. While higher concentrations of jasmonates can be beneficial, it has been surprisingly found that even very low concentrations of jasmonates produce the advantageous properties of the compositions disclosed herein. In some embodiments, the jasmonates of the composition comprise jasmonic acid. In some embodiments, the concentration of jasmonates is 0.001 to 1 ng/ml, 0.001 to 0.1 ng/ml, or 0.001 to 0.004 ng/ml. In some embodiments, these concentrations are prior to a dilution into water or aqueous media before use.

Such compositions as those described above have many advantages for plants grown in various growing media, including soils. They improve cation availability and transport, improve the production of siderophores and thereby elicit siderophore effects (including, but not limited to, scavenging and transport of iron for microbes and crops/plants, especially in soils with high pH and crops/plants containing limited iron micronutrient), improve microbial and fungal biodiversity, and improve utilization efficiency of phytochemicals (including primary and secondary metabolites) by soil microbiome and crops/plants. Additionally, such compositions improve mycorrhizal colonization, increase mycorrhizal spore count, increase several beneficial soil microbial populations, including, but not limited to, beneficial bacteria and actinomycetes.

Any of the abovementioned compositions can be derived from algae. It is appreciated that where the above composition is not derived from algae, it could be reconstituted by dilution of the relevant constituents under controlled conditions into an appropriate diluent (e.g., water or aqueous media). In some embodiments, the algae are macroalgae, such as seaweed or seaweed extract. In some embodiments, the algae are microalgae, such as those from the genus Chlorella, Spirulina, Nannochloropsis and/or Scenedesmus.

Such algal strains can include but are not limited to:
Chlorella vulgaris Beijerinck 9765™ (ATCC 9765 (Strain designation L756a))
Chlorella sp. 50258™ (ATCC 50258 (Strain designation NC64A)
Chlorella vulgaris Beijerinck 30821™ (ATCC 30281)
Chlorella vulgaris var. viridis Chodat 16487™ (ATCC 16487 (Strain designation UTEX 30))
Scenedesmus communis Hegewald 30428 UTEX 76 [CCAP-276/4b]
Scenedesmus obliquus (Turpin) Kutzing 11457 [UTEX 72]
Scenedesmus obliquus (Turpin) Kutzing 11477 Fager D-3 PB6A
Scenedesmus obliquus (Turpin) Kutzing 30580 UTEX 72 [CCAP-276/3a]
Scenedesmus obliquus (Turpin) Kutzing 30431 UTEX 79 [CCAP-276/1b]
Scenedesmus obliquus (Turpin) Kutzing 30430 UTEX 78 [CCAP-276/3b]
Scenedesmus obliquus (Turpin) Kutzing 30434 UTEX 83 [CCAP-276/1a]
Scenedesmus obliquus (Turpin) Kutzing 14477 UTEX 74
Scenedesmus quadricauda (Turpin) Brebisson 11460 [UTEX 76]
Scenedesmus sp. 11464 Pringsheim Z-14-4-1 BAD [Z-14-4-1 BAD]
Spirulina major (UTEX LB 552)
Spirulina maxima (UTEX LB 2342)
Spirulina platensis (ATCC 53844 (Strain designation M-185))
Spirulina platensis subsp. siamese (ATCC 53843)
Spirulina platensis (UTEX LB 2340)
Spirulina platensis (UTEX LB 1926)
Spirulina platensis (UTEX LB 1928)
Spirulina sp. (UTEX LB 2179)
Spirulina sp. (UTEX SP49)
Spirulina sp. (UTEX LB 770)
Spirulina subsalsa (UTEX LB 1954)
Spirulina subsalsa f. versicolor (UTEX LB 1318)
Nannochloropsis sp. (KMMCC-290)

In some embodiments, the compositions disclosed herein can further comprise cultured and enhanced sterilized water. In this context, cultured means water in which a broth of biologically active algae has interacted and decayed, expressing secretomes into the water prior to the removal of the algal biomass. Enhanced sterilized water means that the water has received one or more sterilization treatments (e.g., micro air bubble or ozone treatment), which results in the optimized distribution of nutrients and injected CO2. Furthermore, such water can be enhanced with microbubbles injected therein which increases the mixing and agitation of the various constituents of the broth, which in turns increases the productivity of the culture therein.

In some embodiments, the compositions disclosed herein comprise biomass, ethylene, abscisic acid, brassinolides, or combinations thereof. In some embodiments, the compositions disclosed herein does not comprise biomass, ethylene, abscisic acid, brassinolides, or combinations thereof. The inclusion of biomass, including but not limited to cells, cell parts and cellular debris other than secretomes and/or exudates, in the composition is thought to cause acidification of the soils to which it is applied, which will alter the pH of the soil and can be detrimental to plants which grow best at certain pH ranges. As such, in some embodiments, the absence of biomass confers the benefit that the pH level of the soil is not disturbed upon application of the composition lacking said biomass. Ethylene is known to cause the ripening of fruits, and while this is beneficial in some contexts, in some embodiments, ethylene does not form part of the composition. Abscisic acid is known to promote abscission and dormancy in seeds and buds. In some embodiments, abscisic acid is not form part of the present compositions. In some embodiments, the composition does not comprise brassinolides. Brassinolides are known to be involved in various plant processes such as elongation, flowering, and cell division.

By way of non-limiting examples, plants that can be benefitted by growing using the compositions and methods disclosed herein include any of the following families: Actinidiaceae, Adoxaceae, Allaceae, Anacardiaceae, Apiaceae (Umbelliferae), Arecaceae, Asteraceae, Bromeliaceae, Cactaceae, Caesalpinioideae, Cannabaceae, Capparaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Rosaceae, Rutaceae, Sapindaceae, Saxifragaceae, Solanaceae, and/or Vitaceae.

In some embodiments, the plants growing with mycorrhizal inoculant and/or MHB and/or PGPR/PGPB treatments include but are not limited to strawberries, blueberries, watermelon, tomatoes, tree nuts (e.g., almonds), soybeans, corn (also referred to as maize), wheat, table grapes, wine grapes, leeks, lettuce, potatoes, sugar beets, sugarcane, turf, cannabis, hemp, avocado, carrot, chili peppers, green peppers, citrus, rice and/or spinach.

In further aspects, disclosed herein are methods of enhancing efficiency of mycorrhizal fungi, mycorrhizal soil and/or root inoculation, and/or MHB and/or PGPR/PGPB products on plant growth and yield, comprising providing a composition disclosed herein, or any embodiments thereof disclosed herein, and applying an effective amount of the composition to one or more seeds, soil, growing media, and/or a cultivated area, or any combination thereof. The growing media can comprise bark, clay, coir pith, green compost, grow foam, black peat, white peat, perlite, rice hulls and/or wood fibers. A cultivated area is defined as an area that is cropped every season for seasonal crops (e.g., rice, maize, wheat, soybeans, and the like). In the case of longer season crops like sugarcane or perennial crops like fruit trees, the cultivated area is cropped every year. In some embodiments, the cultivated area can be tilled.

While higher concentrations of each of the constituents of the composition are thought to produce the benefits associated with the compositions of the present invention, it has been surprisingly found that even very low concentrations of each constituent maintain this beneficial effect. In some embodiments, the composition comprises the composition comprises the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01-1 ng/ml, the one or more auxins are at a concentration of 0.001 to 0.5 ng/ml, the salicylic acid is at a concentration of 0.5-1 ng/ml, and/or the one or more jasmonates are at a concentration of 0.001 to 0.1 ng/ml; the one or more gibberellins are at a concentration of to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml; or any concentration or ranges of concentrations therein, and the method further comprises diluting the composition to 25:1 to 150:1, 50:1 to 150:1, or 100:1 ratio of water (or aqueous media) to composition (water:composition) prior to applying the effective amount. For example, the above dilution range would be 25 to 150 parts water to 1 part of the composition disclosed herein. In some embodiments, the diluent is water or other aqueous media.

In some embodiments, the composition is applied to mycorrhizal fungi, mycorrhizal soil and/or root inoculants, MHB, and/or PGPR/PGPB treated soil or plants, growing medium and/or a cultivated area and the effective amount is 0.5 to 1 gallon (1.89 to 3.79 liters, respectively) per acre per week. This is thought to be a particularly advantageous range of volumes at which the composition enhances the mycorrhizal fungi, mycorrhizal soil and/or root inoculant, MHB, and/or PGPR/PGPB benefits, while minimizing water use.

In this context, injection is a method of applying the composition through an irrigation system using water to distribute the composition.

In-furrow is a method of spraying over the seeds in the open seed furrow during planting and can be combined with banded application.

Furrow irrigation is a method of laying out the water channels in such a way where gravity limits the amount of water (or composition) to a suitable amount for the growth of the crop, usually made by the planned placement of ridges and furrows.

Drip irrigation is a type of micro-irrigation system that saves water and nutrients by allowing the composition to drip slowly proximal to the roots of plants either from above the soil surface or buried below the surface. This method minimizes evaporation, and the composition is distributed through a network of valves, pipes, tubing, and emitters.

Fertigation is the injection of fertilizers into an irrigation system. It is closely related to chemigation, where chemicals are injected using an irrigation system. Either term can be used with the present method of applying the composition.

Seed treatment means that seeds are treated with the compositions disclosed herein and/or in a tank mix with other seed treatable products prior to planting. Seed treatment using the composition is thought to be particularly advantageous as it can improve germination and avoids unnecessary wastage of the composition compared to in-soil application where composition can be wasted on weeds, crop residue and soil in which no root systems will develop.

Seed placement means that the composition is placed into the ground proximal to the seed, and thus closer to the root system once it develops.

Further aspects provides methods of enhancing efficiency of mycorrhizal fungi, mycorrhizal soil and/or root inoculation, and/or MHB and/or PGPR/PGPB products on plant growth and yield, comprising providing an algae culture and air to an algae culture broth composition producing system, the system comprising: a sterilizer, an automatic carbon dioxide supply device to promote photosynthesis, an at least partially sealed or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration, and a high-efficiency harvesting device using hollow fiber membranes, and harvesting the composition by separating the composition from biomass of the algae culture using the harvesting device, optionally comprising dehydrating the composition for storage and transport and diluting it prior to use, wherein the method comprises applying an effective amount of the composition to one or more seeds, soils, growing media, and/or a cultivated area.

Such a system could be implemented in a variety of ways using any number of devices. For example, see WO2018009575A1, which is incorporated herein by reference in its entirety, which demonstrates one example system for achieving the compositions disclosed herein. In some embodiments, the sterilizer is used to sterilize the diluent (e.g., water or aqueous media), and comprises any one or more of the following devices or techniques for doing so, or any combination thereof: a fine filtration device, an ultraviolet light emitting device, a reverse osmosis device, a nano/micro-bubble device, an oxygen delivery device, and/or an ozone delivery device. The use of bubbles through the diluent causes solids to rise to the surface for removal, and the introduction of ozone creates hydroxyl radicals (OH—) which sterilize the diluent efficiently.

The at least partially sealed or fully sealed vertical photobioreactor allows for optimal light penetration into the culture which increases productivity thereof. The harvested algae is passed through hollow-fiber membranes to separate the biomass (algal cells and debris) from the compositions disclosed herein.

In any of the preceding aspects or embodiments, the composition can be derived from a microalgae culture comprising Chlorella, Spirulina, Nannochloropsis and/or Scenedesmus. It will be appreciated that any of the embodiments relating to the composition and/or the method of application relevant to the first two aspects are equally applicable to this third aspect.

In further aspects, disclosed herein are methods of enhancing the efficiency of mycorrhizal fungi, mycorrhizal soil and/or root inoculation, and/or MHB products and/or PGPR/PGPB on plant growth and yield, comprising providing an algae culture and air to an algae culture broth composition producing system, the system comprising: a sterilizer; an automatic carbon dioxide supply device to promote photosynthesis; an at least partially sealed or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and a high-efficiency harvesting device using hollow fiber membranes; and harvesting the composition by separating the composition from biomass of the algae culture using the harvesting device, optionally substantially dehydrating the composition for storage and transport, and diluting it prior to use, and applying an effective amount of the composition to one or more seeds, soil, growing media, and/or a cultivated area.

Any of the above mentioned aspects can be combined with any of the embodiments outlined relating to aspects of the composition and methods related to mycorrhizal fungi and/or MHB treated seeds, plants, or soil. As used herein, cations include but are not limited to iron, potassium, phosphorous, zinc, manganese, aluminum, magnesium, and/or combinations thereof. The seeds, plant, and soil can be tested using the same protocols as outlined above (for example, the Haney Soil Health Tool and empirical evidence). The compositions and methods disclosed herein improve the efficiency of mycorrhizal fungi, mycorrhizal soil and/or root inoculation, and/or MHB and/or PGPR/PGPB products on plant growth and yield, and as such has beneficial effects on the health of the plants and soil. In some embodiments, the composition improves the uptake of nutrients in the plants grown in mycorrhizal fungi, mycorrhizal inoculant, and/or MHB and/or PGPR/PGPB treated soil in which the plants are grown. In some embodiments, the composition improves the soil health of the mycorrhizal fungi, mycorrhizal inoculant, and/or MHB and/or PGPR/PGPB treated soil in which the plants are grown. The compositions and methods disclosed herein can enhance soil native plant growth promoters that have the potential to promote uptake of nutrients and cations such as but not limited to phosphorus (P), potassium (K) and iron (Fe), as well as phytohormone production (all together or individually, in different balance depending of the soil microbiome status before adding the product). The compositions and methods disclosed herein can improve both nutrient and cation cycling, mobilization, regulation, and uptake, especially, but not limited to, phosphorus (P), potassium (K) and iron (Fe), as an example. The compositions and methods disclosed herein can also promote the growth of plant growth-promoters (PGP) and plant growth promoting rhizobacteria (PGPR) on the field, which stimulate phytohormone production and stress adaptation. The compositions and methods disclosed herein can also increase mycorrhizal spore counts and colonization, soil microbial populations (including beneficial bacteria and Actinomycetes), supportive soil responses, and increase crop yield.

Disclosed herein are uses of a composition to enhance the efficiency of mycorrhizal fungi, mycorrhizal soil and/or root inoculation, and/or MHB and/or PGPR/PGPB products on plant growth and yield, the composition comprising one or more gibberellins and/or one or more auxins and/or salicylic acid and/or one or more jasmonates.

In some embodiments, the composition comprises one or more gibberellins, one or more auxins salicylic acid, and one or more jasmonates.

In some embodiments, prior to an optional dilution at the point of application to the soil, the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01 to 1 ng/ml, the one or more auxins are at a concentration of 0.001 to 0.5 ng/ml, the salicylic acid is at a concentration of 0.5 to 1 ng/ml, and/or the one or more jasmonates are at a concentration of 0.001 to 0.1 ng/ml; the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml; or any concentrations or ranges of concentrations therein.

In some embodiments, the effective amount of the composition in (a) is applied mycorrhizal fungi, mycorrhizal soil or root inoculant, MHB, and/or PGPR/PGPB treated soil, growing media, and/or the cultivated area, and the effective amount is 1 to 5 liters, 1.89 to 3.79 liters (0.5 to 1 gallon), or 1 to 2 gallons per acre.

In further embodiments, the method further comprises diluting the composition in (a) to 50:1 to 300:1, 100:1 to 250:1, or 150:1 to 200:1 prior to applying the effective amount to the soil and performing one or more in a water:composition ratio, in water prior to performing one or more additional applications of the effective amount of the composition. In some embodiments the diluent is water or other aqueous media.

In some embodiments, the one or more additional applications of the compositions in (a) or (b) are performed at least every 1 to 60 days, 1 to 30 days, 1 to 14 days, 1 to 7 days, or any days or ranges of days therein. This is thought to be an advantageous schedule for repeated applications of the composition to improve efficiency of mycorrhizal fungi, mycorrhizal soil and/or root inoculation, and/or MHB and/or PGPR/PGPB products on plant growth and yield.

In some embodiments, the compositions in (a) further comprises cultured and enhanced sterilized water, optionally at 80% to 99% w/w, 90% to 99.5% w/w, or any % or ranges of % therein.

In some embodiments, the effective amount of the composition in (a) is applied to mycorrhizal fungi, mycorrhizal soil and/or root inoculants, MHB, and/or PGPR/PGPB treated soil, growing media, and/or the cultivated area and the effective amount is 1 to 5 liters, 1.89 to 3.79 liters (0.5 to 1 gallon), 1 to 2 gallons, or any volume or ranges therein per acre.

Reference will now be made in detail to embodiments, examples of which are illustrated in the accompanying drawings, wherein like reference numerals refer to like elements throughout. In this regard, the present embodiments can have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### Examples

### Example 1. Preparation of an Algal Composition

An algae culture broth producing system is described in WO2018/009575. The system is further modified, described as follows.

In WO2018/009575, the device for culture broth sterilization comprises a micro air bubble generator; an air compression and pressure equalization device for the injection of carbon dioxide and oxygen from the atmosphere into the culture broth; an air chilling device to maintain suitable culture broth temperature in response to a water temperature being higher than a predetermined maximum temperature; an automatic carbon dioxide supply device to promote photosynthesis; a sealed vertical photobioreactor configured to contain a culture medium inoculated with an algae, the vertical photobioreactor being configured to allow light into the culture medium, block out pollutants and increase dissolved carbon dioxide and oxygen concentration; a high-efficiency harvesting device using hollow fiber membranes (HFMs); and a hot air drying device using the waste heat generated by air compression. The modified device does not contain an air chilling device; closed photobioreactors; and hot air-drying device.

An air chilling device to maintain suitable culture broth temperature in response to a water temperature being higher than a predetermined maximum temperature is replaced with an air dryer without a temperature control device.

A sealed vertical photobioreactor configured to contain a culture medium inoculated with algae, the vertical photobioreactor being configured to allow light into the culture medium, block out pollutants and increase dissolved carbon dioxide and oxygen concentration is replaced with a system that is not closed: each and every tube has a hole that exposes it to the elements.

A high-efficiency harvesting device using hollow fiber membranes (HFMs) without a hot air drying device that uses the waste heat generated by air compression is used.

In WO2018/009575, the air pressurizing device consists of a device to compress carbon dioxide and oxygen in the atmosphere to 10 bar, a device (tank) to equalize air pressure, and pipes and a gas backflow preventing device to inject the compressed gases into the culture broth. In the modified system, a simpler pressurization is used, i.e., comprising an air compressor, holding tanks, and a CO2 tank (with valve regulators). In WO2018/009575, the air chilling device is for maintaining an optimum water temperature for each algae species in response to a water temperature being higher than a predetermined optimum temperature. In the modified system, there is an air dryer which slightly lowers the temperature of the algae but it does not control temperature to suit the algae's needs. The automatic carbon dioxide supply device supplies gaseous carbon dioxide into the culture broth using a pH sensor in order to promote photosynthesis in the rapid growth phase.

In WO2018/009575, the algae culture broth producing apparatus according to Claim 5, wherein the automatic carbon dioxide supply device automatically supplies carbon dioxide when the pH of the culture broth is 7.26 or higher, and automatically cuts off carbon dioxide when the pH of the culture broth is less than 7.26 if the algae is a freshwater algae, and automatically supplies carbon dioxide when the pH of the culture broth is 7.30 or higher, and automatically cuts off carbon dioxide when the pH of the culture broth is less than 7.30 if the algae is a seawater algae. The modified system is set to introduce CO2 at pH 7.8 and close the solenoid valve at pH 7.2

In WO2018/009575, the compressed gas is injected into the bottom of the apparatus to cause gases in air bubble form to rise vertically and cause ripples in the culture broth, keeping algae from attaching to the walls of the photobioreactor, causing the penetration rate of light, an element of photosynthesis, thus increasing the concentrations of the growth factors dissolved carbon dioxide, nitrogen and oxygen to increase and in turn promoting photosynthesis, and oxygen, which is the metabolic product of photosynthesis, is immediately discharged into the atmosphere through a discharge pipe disposed on top of the apparatus. The modified system does not have such a “discharge pipe” installed—there are small holes drilled into the lid of each tube.

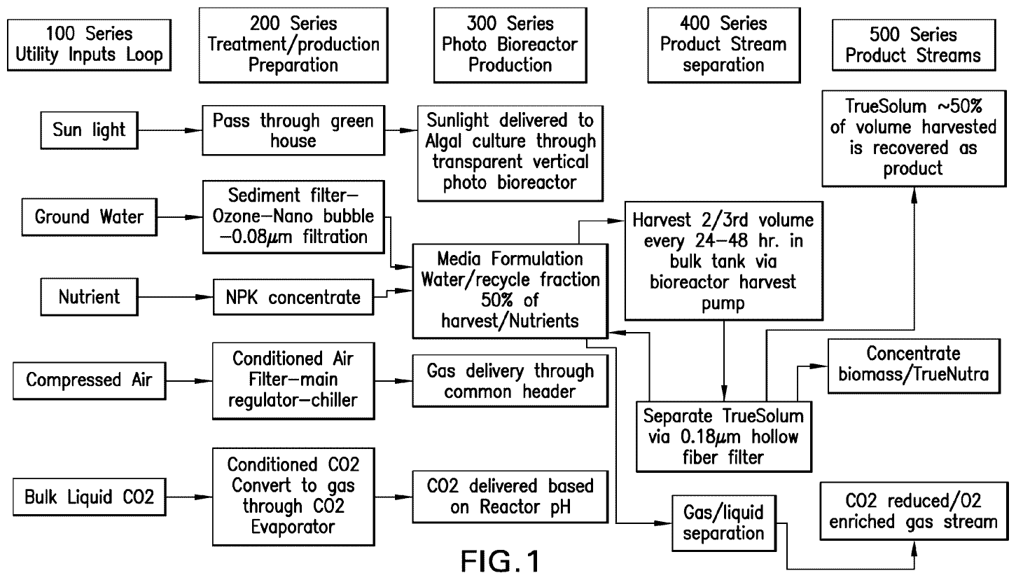

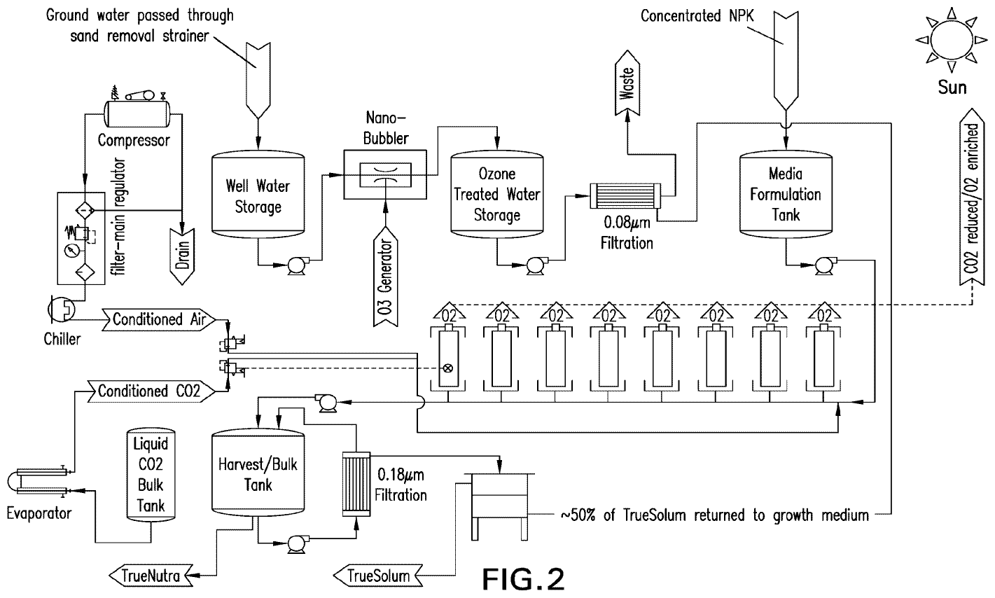

FIG. 1 shows a process flow diagram for producing a composition disclosed herein. FIG. 2 shows an overview of the process for producing a composition disclosed herein.

Using the above-described system, the algal culture and biomass are produced as follows. Water is sanitized using several levels of purification: this redundancy ensures that no potentially harmful organisms remain in the water. A mixture of ozone, high oxygen levels, and microbubbles is dispersed throughout well water and allowed to rest until the gases release from the water itself leaving a sanitized final solution. The final solution is passed through an ultrafiltration hollow fiber membrane to remove any debris or sediment remaining before going into the system.

CO2 is stored in a large liquid CO2 tank outside. It passes through a vaporizer to ensure even release of gas even in extreme temperatures, and then passes through a primary regulator before being regulated again at the divide between the north and south halves of the greenhouse.

Air is generated through rotary screw compressors and stored in large storage tanks to maintain constant air pressure in the system. Before the air is introduced to the PBRs (photobioreactors) it is passed through a dryer, as well as through a final near high-efficiency particulate air (HEPA) level of filtration, and a large regulator to ensure consistent pressure and that too much pressure is not placed on air fittings.

Small levels of liquid fertilizer are mixed into a solution of either water or algae culture medium without biomass product for reintroduction to the PBRs.

The photobioreactors are connected to the system through a main drain/fill tube at the bottom of each unit. Using these we can fill them with sanitized water or an NPK solution, as well as drain the lots into the main tank for harvest filtration.

Each of the units is connected to both air and CO2. The air is used mostly for even dispersal of the algal culture for optimal sunlight absorption, CO2 dispersal, and general agitation. After they are mixed, the air and the CO2 are regulated a final time at each individual group of PBRs.

Using small pore size hollow fiber membranes, the biomass is removed from the culture broth and separated out as the final product is pumped into a storage tank with little to no algae remaining.

### Example 2. Application of the Compositions to Tomato Plants—Florida (Trial #1 of 2

A research study in two trials was performed to assess if microalgae culture medium without biomass product is affected to tomato plant (FL47 variety) soil beneficial microorganisms of bacteria and fungi biodiversity composition and population, as well as enhancing plant productivity, growth, and crop yield under simulated grower conditions. For this experiment, the microalgae culture medium without biomass was applied weekly approximately 4.5 weeks (32 days) after planting and transplantation for a total of 9 applications at one geographical location in the United States (Florida Ag Research in Thonotosassa, Florida) with four replicates and five different treatments in the following soil conditions: 94.4% sand, 2.2% silt, 3.4% clay.

In this experiment, the untreated lot was maintained using a standard grower's protocol regarding farming, supplying water, and fertilizers. The treated lots were maintained using exactly the same protocol, with the exception of having the microalgae culture medium without biomass applied 9 times weekly at a dosage of 0.5 gallon/acre, diluted into 250 gallons of water and applied diluted water volume for drip trailer the first 4 applications (5/9, 5/15, 5/22, 5/28) and the remaining 5 for drip tape (6/5, 6/11, 6/19, 6/27, 7/2) irrigation system, with the AMF product (MycoApply's EndoMaxx) being applied once (5/9) either with the NPK fertilizer (4-2-9) or NPK fertilizer and TrueSolum (algae composition disclosed herein comprising one or more gibberellins, one or more auxins, salicylic acid, and/or one or more jasmonates).

For this experimental study, water, NPK fertilizer, AMF product, and the algal derived microalgae culture medium without biomass was supplied to soil via drip irrigation (either drip trailer or drip tape irrigation systems depending on the timing).

The various treatments and associated applications are shown in Tables 1 and 2.

TABLE 1

| Trt | | | Rate | Appl | Amount |
|---|---|---|---|---|---|
| No. | Treatment Name | Rate | Unit | Amount | Unit |
| 1 | 4-2-9 | 34.62 | gal/a | 3500 | GAL/AC |
| 2 | Chlopia | 0.5 | gal/a | 3500 | GAL/AC |
| | 4-2-9 | 8.655 | gal/a | 3500 | GAL/AC |
| 3 | Chlopia | 0.5 | gal/a | 3500 | GAL/AC |
| 4 | MycoApply's EndoMaxx | 6 | g/a | 3500 | GAL/AC |
| | 4-2-9 | 34.62 | gal/a | 3500 | GAL/AC |
| 5 | MycoApply's EndoMaxx | 6 | g/a | 3500 | GAL/AC |
| | Chlopia | 0.5 | gal/a | 3500 | GAL/AC |
| | 4-2-9 | 34.62 | gal/a | 3500 | GAL/AC |

TABLE 2
Applications:

| | |
|---|---|
| A-May 9, 2020 | Drip Trailer, 3500 GPA, 10 PSI |
| B-May 15, 2020 | Drip Trailer, 3500 GPA, 10 PSI |
| C-May 22, 2020 | Drip Trailer, 3500 GPA, 10 PSI |
| D-May 28, 2020 | Drip Trailer, 3500 GPA, 10 PSI |
| E-Jun. 5, 2020 | Drip Tape, 3500 GPA, 10 PSI |
| F-Jun. 11, 2020 | Drip Tape, 3500 GPA, 10 PSI |
| G-Jun. 19, 2020 | Drip Tape, 3500 GPA, 10 PSI |
| H-Jun. 27, 2020 | Drip Tape, 3500 GPA, 10 PSI |
| I-Jul. 2, 2020 | Drip Tape, 3500 GPA, 10 PSI |

FL47 tomatoes were transplanted 19″ apart on 6′-wide beds in sandy, drip irrigated, soil, with plots that were 30′ on length and 6′ width and replicated four times. MycoApply's EndoMaxx product that was applied contained the following mycorrhizae fungal strains as active ingredients: Glomus aggregatum, Glomus etunicatum, Glomus intraradices, Glomus mosseae.

Plants were sacrificed July 11 and root characteristics were recorded. Root vigor (Table 3), rated on a 0-100 scale, was significantly higher on the plants treated with MycoApply. Root colonization by mycorrhizae was enhanced by TrueSolum applied together with EndoMaxx and 4-2-9, consistent with higher spore counts (Table 4). Bacterial CFU counts per gram of soil were also higher for plots treated with EndoMaxx, 4-2-9 and TrueSolum, but also for TrueSolum applied solo. Actinomycete counts were lowest on the plots which received no TrueSolum (Table 4).

The results of the various treatments of the study on root vigor are shown in Table 3, showing statistically significant improvement (LSD, P=0.05) over the other treatments.

As used herein in the examples, LSD (Least Significant Difference) is the value at a particular level of statistical probability (e.g., P≤0.01—means with 99% accuracy) when exceeded by the difference between two varietal means for a particular characteristic, then the two varieties are said to be distinct for that characteristic at that or lesser levels of probability. Means followed by the same letter or symbol do not significantly differ (P=0.05, LSD).

TABLE 3
Root Vigor

| Trt | | | Rate | |
|---|---|---|---|---|
| No. | Treatment Name | Rate | Unit | Jul. 11, 2020 |
| 1 | 4-2-9 | 34.62 | gal/a | 76.80 b |
| 2 | Chlopia | 0.5 | gal/a | 78.30 ab |
| | 4-2-9 | 8.655 | gal/a | |
| 3 | Chlopia | 0.5 | gal/a | 77.00 b |
| 4 | MycoApply EndoMaxx | 6 | g/a | 81.80 a |
| | 4-2-9 | 34.62 | gal/a | |
| 5 | MycoApply EndoMaxx | 6 | g/a | 82.50 a |
| | Chlopia | 0.5 | gal/a | |
| | 4-2-9 | 34.62 | gal/a | |

The results of the various treatments of the study on root colonization by mycorrhizae and other microbial populations of bacteria and actinomycetes are shown in Table 4, showing statistically significant improvement (LSD, P=0.05) over the other treatments for mycorrhizal root colonization and sport count, and statistical improvement of microbial population improvement over the other treatments, with Treatment 5 showing approximately 25% over Treatment 1 (Control) for root colonization by mycorrhizae, which is consistent with higher spore counts. Additionally, bacterial CFU counts per gram of soil were also higher for Treatment 5 plots by over 343% over Treatment 1 (Control), but also for Treatment 3 (TrueSolum Only) by 317% over Treatment 1. TrueSolum used in Treatment 3 and Treatment 5 also increased Actinomycete counts by 281% and 242%, respectively, versus Treatment 1 (Control).

TABLE 4
Root and soil analysis for root colonization by mycorrhizae,
and bacteria and Actinomycete counts in soil (Jul. 11, 2020)

| | | Microbial Analysis | | | |
|---|---|---|---|---|---|
| | | Mycorrhizal | | | |
| Trt | Treatment | % | Count/ | Bacteria | Actinomycetes |
| No. | Name | Colonization | 100 g | CFU/g | Total/g |
| 1 | 4-2-9 | 0.0% c | 0.00 c | 14.50 c | 7.30 c |
| 2 | Chlopia | 1.25% c | 1.30 c | 48.00 b | 20.80 b |
| | 4-2-9 | | | | |
| 3 | Chlopia | 0.0% c | 0.00 c | 60.50 a | 27.80 a |
| 4 | MycoApply | 11.02% b | 45.00 b | 12.80 c | 8.80 c |
| | EndoMaxx | | | | |
| | 4-2-9 | | | | |
| 5 | MycoApply | 24.87% a | 81.50 a | 64.30 a | 25.00 ab |
| | EndoMaxx | | | | |
| | Chlopia | | | | |
| | 4-2-9 | | | | |

RapidSCAN remote sensing equipment was used to measure canopy density and greenness (Normalized Difference Red Edge, NDRE, and Normalized Difference Vegetative Index, NDVI, respectively) four times. Plots treated with 4-2-9 had significantly higher readings after applications C and F (see Table 5A); applying TrueSolum decreased NDRE and NDVI, as shown in Tables 5A and 5B.

The results of the various treatments of the study on NDRE are shown in Table 5A.

TABLE 5A
NDRE

| Trt | Treatment | | Rate | May 14, 2020 | May 27, 2020 | Jun. 12, 2020 | Jun. 28, 2020 |
|---|---|---|---|---|---|---|---|
| No. | Name | Rate | Unit | 5 DA-A | 5 DA-C | 1 DA-F | 9 DA-G |
| 1 | 4-2-9 | 34.62 | gal/a | 0.248 a | 0.309 a | 0.265 a | 0.170 a |
| 2 | Chlopia | 0.5 | gal/a | 0.251 a | 0.256 b | 0.212 b | 0.119 a |
| | 4-2-9 | 8.655 | gal/a | | | | |
| 3 | Chlopia | 0.5 | gal/a | 0.259 a | 0.257 b | 0.207 b | 0.127 a |
| 4 | MycoApply EndoMaxx | 6 | g/a | 0.237 a | 0.277 b | 0.250 a | 0.145 a |
| | 4-2-9 | 34.62 | gal/a | | | | |
| 5 | MycoApply EndoMaxx | 6 | g/a | 0.252 a | 0.279 ab | 0.272 a | 0.162 a |
| | Chlopia | 0.5 | gal/a | | | | |
| | 4-2-9 | 34.62 | gal/a | | | | |

The results of the various treatments of the study on NVDI are shown in Table 5B.

TABLE 5B
NDVI

| Trt | Treatment | | Rate | May 14, 2020 | May 27, 2020 | Jun. 12, 2020 | Jun. 28, 2020 |
|---|---|---|---|---|---|---|---|
| No. | Name | Rate | Unit | 5 DA-A | 5 DA-C | 1 DA-F | 9 DA-G |
| 1 | 4-2-9 | 34.62 | gal/a | 0.655 a | 0.707 a | 0.666 a | 0.506 a |
| 2 | Chlopia | 0.5 | gal/a | 0.645 a | 0.585 a | 0.546 b | 0.346 a |
| | 4-2-9 | 8.655 | gal/a | | | | |
| 3 | Chlopia | 0.5 | gal/a | 0.650 a | 0.602 a | 0.539 b | 0.363 a |
| 4 | MycoApply EndoMaxx | 6 | g/a | 0.607 a | 0.633 a | 0.668 a | 0.414 a |
| | 4-2-9 | 34.62 | gal/a | | | | |
| 5 | MycoApply EndoMaxx | 6 | g/a | 0.651 a | 0.618 a | 0.712 a | 0.469 a |
| | Chlopia | 0.5 | gal/a | | | | |
| | 4-2-9 | 34.62 | gal/a | | | | |

Tomatoes were harvested thrice, where counts and weights of marketable tomatoes were recorded as well count of culls due to sunscald or other unmarketable characteristics. Individual treatments did not yield significantly more or less total fruit of any grade (Table 7), however total marketable yields were showing statistically significant improvement (LSD, P=0.05) over the other treatments from tomatoes treated with MycoApply, 4-2-9, and TrueSolum together (Table 6), where Treatment 5 was 16.7% higher than Treatment 1 (Control) and 19.9% higher than Treatment 4. TrueSolum alone yielded the least marketable tomatoes, as to be expected. The results show that TrueSolum has symbiotic working relationship with mycorrhizae, thus enhancing the beneficial microbial content in the soil. Research has shown that higher microbial activity in the soil promotes better uptake of nutrients by the plant, thus leading to more marketable yield. It is hypothesized that that TrueSolum acts as a prebiotic and/or a signaling compound for the mycorrhizae, thus enhancing the beneficial content in the soil.

The results of the various treatments of the study on total marketable yield per acre are shown in Table 6.

TABLE 6

| Trt | Treatment | | Rate | Yields per Acre | |
|---|---|---|---|---|---|
| No. | Name | Rate | Unit | Marketable Lbs | Estimated $ |
| 1 | 4-2-9 | 34.62 | gal/a | 27697 b | $ 17,671 b |
| 2 | Chlopia | 0.5 | gal/a | 25277 bc | $ 16,127 bc |
| | 4-2-9 | 8.655 | gal/a | | |
| 3 | Chlopia | 0.5 | gal/a | 24630 c | $ 15,714 c |
| 4 | MycoApply | 6 | g/a | 26950 bc | $ 17,194 bc |
| | EndoMaxx | | | | |
| | 4-2-9 | 34.62 | gal/a | | |
| 5 | MycoApply | 6 | g/a | 32310 a | $ 20,614 a |
| | EndoMaxx | | | | |
| | Chlopia | 0.5 | gal/a | | |
| | 4-2-9 | 34.62 | gal/a | | |

The results of the various treatments of the study on total yield composition are shown in Table 7.

TABLE 7

| | | Yield Composition (by count) | | | | |
|---|---|---|---|---|---|---|
| Trt | Treatment | Extra | | | Cull- | Cull- |
| No. | Name | Large | Large | Medium | Sunscald | Other |
| 1 | 4-2-9 | 7.31% a | 40.30% a | 40.68% a | 7.02% a | 4.69% a |
| 2 | Chlopia | 3.85% a | 44.80% a | 39.59% a | 6.85% a | 4.90% a |
| | 4-2-9 | | | | | |
| 3 | Chlopia | 2.87% a | 42.26% a | 42.58% a | 7.78% a | 4.51% a |
| 4 | MycoApply EndoMaxx | 6.29% a | 39.07% a | 43.71% a | 6.53% a | 4.41% a |
| | 4-2-9 | | | | | |
| 5 | MycoApply EndoMaxx | 5.77% a | 43.16% a | 39.55% a | 6.65% a | 4.86% a |
| | Chlopia | | | | | |
| | 4-2-9 | | | | | |

Overall, with the objective of this study to determine TrueSolum's (as also called Chlopia herein) efficacy in enhancing tomato growth, marketable yield, and ability to supplement and/or enhance grower standard program fertility and arbuscular mycorrhizae fungi (AMF) when applied together, the study found that when applied together with commercial AMF product MycoApply's EndoMaxx and 4-2-9 NPK fertilizer, TrueSolum significantly improved marketable tomato yields, and statistically improved and enhanced root vigor, mycorrhizal root colonization and spore count, and soil microbial populations, including bacterial CFU and Actinomycete counts, compared to grower's standard program (GSP), GSP+mycorrhizae, and TrueSolum alone.

The results show that TrueSolum has a symbiotic working relationship with mycorrhizae and can enhance and drastically boost overall microbial activity in the soil. It appears that TrueSolum functions as a prebiotic and/or signaling compound to the mycorrhizae, thus improving the value of the mycorrhizae, the quality of the soil, and the availability of nutrients for uptake by the plant.

### Example 3. Application of the Compositions to Tomato Plants—Florida (Trial #2 of 2

A two trial research study was performed to assess if microalgae culture medium without biomass product is affected tomato plant (FL47 variety) soil beneficial microorganisms of bacteria and fungi biodiversity composition and population, as well as enhancing plant productivity, growth, and crop yield under simulated grower conditions. For this experiment, the microalgae culture medium without biomass was applied every 10 days approximately beginning at planting via transplantation for a total of 5 applications at one geographical location in the United States (Florida Ag Research in Thonotosassa, Florida) with four replicates and five different treatments in the following soil conditions: 94.4% sand, 2.2% silt, 3.4% clay.

In this experiment, the untreated plots were maintained using a standard grower's protocol regarding farming, supplying water, and fertilizers. The treated plots were maintained using exactly the same protocol, with the exception of having the microalgae culture medium without biomass applied every 10 days at a dosage of 1.0 gallon/acre, diluted into 250 gallons of water and applied diluted water volume for drip irrigation system (5/26 as soil pretreatment, 6/3 on planting day, 6/13 at 10 Days After Planting [DAP], 6/23 at 20 DAP, 7/3 at 30 DAP) with the AMF product (MycoApply's EndoMaxx Liquid) being applied once (5/26) either with the NPK fertilizer (4-2-9) or NPK fertilizer and TrueSolum.

For this experimental study, water, NPK fertilizer, AMF product, and the algal derived microalgae culture medium without biomass was supplied to soil of all plots via drip irrigation.

The various treatments and design of the study are shown in Table 8.

TABLE 8

| Trt | Treatment | | Rate | Appl | Amount |
|---|---|---|---|---|---|
| No. | Name | Rate | Unit | Amount | Unit |
| 1 | 4-2-9 | 34.62 | gal/a | 3500 | GAL/AC |
| 2 | Chlopia | 1.0 | gal/a | 3500 | GAL/AC |
| | 4-2-9 | 8.655 | gal/a | 3500 | GAL/AC |
| 3 | Chlopia | 1.0 | gal/a | 3500 | GAL/AC |
| 4 | MycoApply's | 92 | ml/a | 3500 | GAL/AC |
| | EndoMaxx Liquid | | | | |
| | 4-2-9 | 34.62 | gal/a | 3500 | GAL/AC |
| 5 | MycoApply's | 92 | ml/a | 3500 | GAL/AC |
| | EndoMaxx Liquid | | | | |
| | Chlopia | 1.0 | gal/a | 3500 | GAL/AC |
| | 4-2-9 | 34.62 | gal/a | 3500 | GAL/AC |

FL47 tomatoes were transplanted 19″ apart on 6′-wide beds in sandy, drip irrigated, soil, with plots that were 30′ on length and 6′ width and replicated four times. MycoApply's EndoMaxx product that was applied contained the following Mycorrhizae fungal strains as active ingredients: Glomus aggregatum, Glomus etunicatum, Glomus intraradices, Glomus mosseae.

Sampling occurred 3 times for each treatment (after 21 days, 30 days, and 45 days) and plants were later sacrificed, with both production yield and mycorrhizae characteristics recorded. Total marketable yield increased and root colonization by mycorrhizae was enhanced by TrueSolum applied together with EndoMaxx and 4-2-9, consistent with higher spore counts (Table 9).

TABLE 9
Trial Results

| | | Mycorrhizal Analysis | | | |
|---|---|---|---|---|---|
| Trt | Treatment | % Spore Population | | % Root Colonization | |
| No. | Name | (30 Days) | (45 Days) | (30 Days) | (45 Days) |
| 1 | 4-2-9 (UTC) | 0.0% | 0.0% | 0.0% | 0.0% |
| 2 | Chlopia | 0.0% | 0.0% | 0.0% | 0.0% |
| | 4-2-9 | | | | |
| 3 | Chlopia | 0.0% | 0.0% | 0.0% | 0.0% |
| 4 | MycoApply | 117% | 107% | 146% | 131% |
| | EndoMaxx | | | | |
| | 4-2-9 | | | | |
| 5 | MycoApply | 137% | 129% | 218% | 142% |
| | EndoMaxx | | | | |
| | Chlopia | | | | |
| | 4-2-9 | | | | |

Tomatoes were harvested and marketable yield was recorded. Total marketable production yields of plots treated with MycoApply, 4-2-9, and TrueSolum together showed numeric improvement over the Control: specifically, Treatment 5 was 182% higher than Treatment 1 (UTC).

Overall, with the objective of this study to determine TrueSolum's (formerly known as Chlopia) efficacy in improving tomato marketable yield and ability to supplement and/or enhance grower standard program fertility and arbuscular mycorrhizae fungi (AMF), the study found that TrueSolum when applied together with commercial AMF product MycoApply's EndoMaxx and 4-2-9 fertilizer, significantly improved marketable tomato yields, as well as improved mycorrhizal root colonization and spore count, compared to grower's standard program (GSP), GSP+mycorrhizae, and TrueSolum alone.

The results show that TrueSolum has a symbiotic working relationship with mycorrhizae and can enhance and drastically boost overall microbial activity in the soil. Similar to Trial #1, it appears that TrueSolum functions as a prebiotic and/or signaling compound to the mycorrhizae, thus improving the value of the mycorrhizae, the quality of the soil, and the availability of nutrients for uptake by the plant.

### Example 4. Application of the Compositions to Variety of Tomato Plants—California

A research study was performed to assess if microalgae culture medium without biomass product is affected to tomato plant (Q27 variety) soil beneficial microorganisms of bacteria and fungi biodiversity composition and population, as well as enhancing plant productivity, growth, and crop yield under simulated grower conditions. For this experiment, the microalgae culture medium without biomass was applied weekly approximately 8 weeks (56 days) after planting and transplantation for a total of 9 applications at one geographical location in the United States (Pacific Ag Research, Arroyo Grande, California) with four replicates and 4 different treatments in the following soil conditions: 37% sand, 29% silt, 34% clay.

In this experiment, the untreated plots were maintained using a standard grower's protocol regarding farming, supplying water, and fertilizers. The treated lots were maintained using exactly the same protocol, with the exception of having the microalgae culture medium without biomass applied 9 times weekly at a dosage of 1.0 gallon/acre (drip) and 0.6 liter/acre (foliar) on an alternating drip-foliar spray program, diluted into 250 gallons of water and applied diluted water volume for drip application these 5 applications (8/31, 9/14,9/28, 10/12, 10/26) and the remaining 4 for foliar application [4 nozzles, TG-0.5 Fullcone nozzles, 50 PSI](9/8, 9/21, 10/05, 10/19) irrigation system, with the AMF product (MycoApply's EndoMaxx) being applied once (8/31) either with the NPK fertilizer or NPK fertilizer and TrueSolum.

For this experimental study, water, NPK fertilizer, AMF product, and the algal derived microalgae culture medium without biomass supplied to soil of all lots was via drip irrigation.

The various treatments and associated applications of the study are shown in Tables 10 and 11.

TABLE 10

| Trt | Treatment | Form | | Rate | Appl | Amount | Mix | Mix |
|---|---|---|---|---|---|---|---|---|
| No. | Name | Type | Rate | Unit | Amount | Unit | Size | Unit |
| 1 | Growers Standard (GSP) | | | | | | | |
| 2 | GSP + Chlopia drip | L | 1 | gal/a | 0.125 | acre inch | 147 | L |
| | GSP + Chlopia foliar | L | 0.6 | l/a | 40 | GAL/AC | 1.91 | L |
| 3 | GSP + Chlopia drip | L | 1 | gal/a | 0.125 | acre inch | 147 | L |
| | GSP + Chlopia foliar | L | 0.6 | l/a | 40 | GAL/AC | 1.91 | L |
| | MycoApply EndoMaxx | D | 6 | g/a | 0.125 | acre inch | 147 | L |
| 4 | MycoApply EndoMaxx | D | 6 | g/a | 0.125 | acre inch | 147 | L |

TABLE 11
Applications:

| | |
|---|---|
| A - Aug. 31, 20: | Drip |
| B - Sep. 8, 20: | Foliar, 4 Nozzles, TG-0.5 Fullcone nozzles, 50 PSI |
| C - Sep. 14, 20: | Drip |
| D - Sep. 21, 20: | Foliar, 4 Nozzles, TG-0.5 Fullcone nozzles, 45 PSI |
| E - Sep. 28, 20: | Drip |
| F - Oct. 5, 20: | Foliar, 4 Nozzles, TG-0.5 Fullcone nozzles, 50 PSI |
| G - Oct. 12, 20: | Drip |
| H - Oct. 19, 20: | Foliar, 4 Nozzles, TG-0.5 Fullcone nozzles, 50 PSI |
| I - Oct. 26, 20: | Drip |

Q27 tomatoes were transplanted 18″ apart on 5′-wide beds in sandy, drip irrigated, soil, with plots that were 25′ in length and 5′ width and replicated four times. MycoApply's EndoMaxx product that was applied contained the following Mycorrhizae fungal strains as active ingredients: Glomus aggregatum, Glomus etunicatum, Glomus intraradices, and Glomus mosseae.

Plants were sacrificed July 19 and root characteristics were recorded. Vigor, rated on a 0-10 scale, was higher in the plants treated with MycoApply. The results of the various treatments of the study on vigor are shown in Table 12, showing multiple timepoints where the treatment of GSP+Chlopia+mycorrhizae (Treatment 3) had improved vigor over the Control (Treatment 1).

TABLE 12
Vigor (0-10)

| Trt | Treatment | Sep. 8, 2020 | Sep. 21, 2020 | Oct. 5, 2020 | Oct. 19, 2020 | Nov. 2, 2020 | Nov. 17, 2020 |
|---|---|---|---|---|---|---|---|
| No. | Name | 8 DA-A | 7 DA-C | 7 DA-E | 7 DA-G | 7 DA-I | 22 DA-I |
| 1 | Growers Standard | 5.0 A | 4.8 a | 4.8 a | 5.5 a | 4.3 a | 4.5 a |
| | (GSP) | | | | | | |
| 2 | GSP + Chlopia Drip | 5.5 A | 4.5 a | 5.3 a | 5.8 a | 4.8 a | 4.3 a |
| | GSP + Chlopia Foliar | | | | | | |
| 3 | GSP + Chlopia Drip | 5.8 A | 4.5 a | 5.5 a | 5.8 a | 4.8 a | 4.5 a |
| | GSP + Chlopia Foliar | | | | | | |
| | MycoApply | | | | | | |
| | EndoMaxx | | | | | | |
| 4 | MycoApply | 5.8 A | 5.0 a | 5.3 a | 6.3 a | 5.3 a | 4.8 a |
| | Endo Maxx | | | | | | |

RapidSCAN remote sensing equipment was used to objectively measure canopy (Normalized Difference Vegetative Index [NDVI]) and density (Normalized Difference Red Edge [NDRE]) four times. Both NDVI and NDRE were highest for the Chlopia and MycoApply-treated plots, significantly so at 7 DA-I, as shown in Tables 13A and 13B. Tomatoes treated with Chlopia and/or MycoApply had higher RapidSCAN NDRE and NDVI readings.

The results of the various treatments of the study on NDVI are shown in Table 13A.

TABLE 13A
Normalized Difference Vegetation Index (NDVI, 0-1)

| Trt | Treatment | Sep. 8, 2020 | Sep. 21, 2020 | Oct. 5, 2020 | Oct. 19, 2020 | Nov. 2, 2020 | Nov. 17, 2021 |
|---|---|---|---|---|---|---|---|
| No. | Name | 8 DA-A | 7 DA-C | 7 DA-E | 7 DA-G | 7 DA-I | 22 DA-I |
| 1 | Growers Standard (GSP) | 0.7907 a | 0.7950 a | 0.8041 a | 0.7901 a | 0.7709 b | 0.7747 a |
| 2 | GSP + Chlopia Drip - 1 gal/a | 0.8059 a | 0.8164 a | 0.8368 a | 0.8258 a | 0.8125 a | 0.7842 a |
| | GSP + Chlopia Foliar - 0.6 l/a | | | | | | |
| 3 | GSP + Chlopia Drip - 1 gal/a | 0.8015 a | 0.8181 a | 0.8364 a | 0.8252 a | 0.8104 a | 0.6265 a |
| | GSP + Chlopia Foliar - 0.6 l/a | | | | | | |
| | MycoApply EndoMaxx - 6 | | | | | | |
| | g/a | | | | | | |
| 4 | MycoApply EndoMaxx - 6 | 0.7967 a | 0.8075 a | 0.8281 a | 0.8155 a | 0.8021 a | 0.7885 a |
| | g/a | | | | | | |

The results of the various treatments of the study on NRDE are shown in Table 13B.

TABLE 13B
Normalized Difference Red Edge (NDRE, 0-1)

| Trt | Treatment | Sep. 8, 2020 | Sep. 21, 2020 | Oct. 5, 2020 | Oct. 19, 2020 | Nov. 2, 2020 | Nov. 17, 2021 |
|---|---|---|---|---|---|---|---|
| No. | Name | 8 DA-A | 7 DA-C | 7 DA-E | 7 DA-G | 7 DA-I | 22 DA-I |
| 1 | Growers Standard (GSP) | 0.3382 a | 0.3236 a | 0.3426 a | 0.3320 a | 0.3101 b | 0.2958 a |
| 2 | GSP + Chlopia Drip - 1 | 0.3453 a | 0.3284 a | 0.3609 a | 0.3430 a | 0.3362 a | 0.3070 a |
| | gal/a | | | | | | |
| | GSP + Chlopia Foliar - 0.6 | | | | | | |
| | l/a | | | | | | |
| 3 | GSP + Chlopia Drip - 1 | 0.3487 a | 0.3365 a | 0.3622 a | 0.3503 a | 0.3383 a | 0.2299 a |
| | gal/a | | | | | | |
| | GSP + Chlopia Foliar - 0.6 | | | | | | |
| | l/a | | | | | | |
| | MycoApply EndoMaxx - 6 | | | | | | |
| | g/a | | | | | | |
| 4 | MycoApply EndoMaxx - 6 | 0.3371 a | 0.3235 a | 0.3531 a | 0.3476 a | 0.3305 a | 0.2988 a |
| | g/a | | | | | | |

Tomatoes were harvested four times (10/22, 11/6, 11/19, 11/24), where counts and weights of marketable tomatoes were recorded for each ripeness stage (green, orange, red). Plots treated with Chlopia and Chlopia+Myco Apply Endomaxx yielded more red tomatoes than the grower standard alone (Table 15), however estimated gross returns were uniform (Table 14), indicating that the treated were more mature and thus could be harvested earlier than the Control. The results show that TrueSolum has symbiotic working relationship with mycorrhizae, thus enhancing the beneficial microbial content in the soil. Research has shown that higher microbial activity in the soil promotes better uptake of nutrients by the plant, thus leading to more marketable yield. It is hypothesized that that TrueSolum acts as a prebiotic and/or signaling molecule for the mycorrhizae, thus enhancing the beneficial content in the soil. The results of the various treatments of the study on total marketable yield per acre are shown in Table 14.

TABLE 14
Estimated Yield (25-lb cartons) per Acre and Estimated Gross Returns
($/Acre), calculated using a value of $16 per 25-lb loose carton

| | | ESTIMATED PER ACRE | |
|---|---|---|---|
| Trt | Treatment | | GROSS |
| No. | Name | CARTONS | INCOME |
| 1 | Growers Standard (GSP) | 2681.5 a | $ 42,905 a |
| 2 | GSP + Chlopia Drip - 1 gal/a | 2921.3 a | $ 46,740 a |
| | GSP + Chlopia Foliar - 0.6 l/a | | |
| 3 | GSP + Chlopia Drip - 1 gal/a | 3081.5 a | $ 49,304 a |
| | GSP + Chlopia Foliar - 0.6 l/a | | |
| | MycoApply EndoMaxx - 6 g/a | | |
| 4 | MycoApply EndoMaxx - 6 g/a | 2893.8 a | $ 46,301 a |

The results of the various treatments of the study on total yield composition are shown in Table 15.

TABLE 15
Yield Composition (%)

| Trt | Treatment | YIELD COMPOSITON (%) | | |
|---|---|---|---|---|
| No. | Name | RED | ORANGE | GREEN |
| 1 | Growers Standard (GSP) | 18.59% a | 33.39% a | 48.02% a |
| 2 | GSP + Chlopia Drip - 1 gal/a | 21.09% a | 30.58% a | 48.33% a |
| | GSP + Chlopia Foliar - 0.6 l/a | | | |
| 3 | GSP + Chlopia Drip - 1 gal/a | 21.05% a | 33.86% a | 45.09% a |
| | GSP + Chlopia Foliar - 0.6 l/a | | | |
| | MycoApply EndoMaxx - | | | |
| | 6 g/a | | | |
| 4 | MycoApply EndoMaxx - | 19.93% a | 29.97% a | 50.10% a |
| | 6 g/a | | | |

Overall, with the objective of this study to determine TrueSolum's efficacy in enhancing tomato growth, marketable yield, and ability to supplement and/or enhance grower standard program fertility and arbuscular mycorrhizae fungi (AMF) when applied together, the study found that TrueSolum when applied together with commercial AMF product MycoApply's EndoMaxx and 4-2-9 fertilizer, significantly improved marketable tomato yields, and statistically improved and enhanced root vigor, mycorrhizal root colonization and spore count, and soil microbial populations, including bacterial CFU and actinomycete counts, compared to grower's standard program (GSP), GSP+mycorrhizae, and TrueSolum alone.

The results show that TrueSolum has a symbiotic working relationship with mycorrhizae and can enhance and drastically boost overall microbial activity in the soil. It appears that TrueSolum functions as a prebiotic or signaling compound to the mycorrhizae, thus improving the value of the mycorrhizae, the quality of the soil, and the availability of nutrients for uptake by the plant.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details can be made therein without departing from the spirit and scope of the disclosure as defined by the following claims.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

### REFERENCES

1. G Mamatha1, D J Bagyaraj, S Jaganath. Inoculation of field-established mulberry and papaya with arbuscular mycorrhizal fungi and a mycorrhiza helper bacterium. Mycorrhiza (2002) Dec;12(6):313-6. doi: 10.1007/s00572-002-0200-y.
2. S Garcia, J Racsko. The Role of Arbuscular Mycorrhical Fungi in Plant Nutrition. Greenhouse Product News. https://gpnmag.com/article/the-role-of-arbuscular-mycorrhizal-fungi-in-plant-nutrition/(accessed Dec. 17, 2021).
3. Naheeda Begum1, Cheng Qin1, Muhammad Abass Ahanger1, et al. Role of Arbuscular Mycorrhizal Fungi in Plant Growth Regulation: Implications in Abiotic Stress Tolerance. Front. Plant Sci., 19 Sep. 2019.1 https://doi.org/10.3389/fpls.2019:01068.
4. Zhu, X. C., Song, F. B., Liu, S. Q., Liu, F. L. (2016). Arbuscular mycorrhiza improve growth, nitrogen uptake, and nitrogen use efficiency in wheat grown under elevated CO2. Mycorrhiza 26, 133-140. doi: 10.1007/s00572-015-0654-3.
5. Ahanger, M. A., Tyagi, S. R., Wani, M. R., Ahmad, P. (2014). “Drought tolerance: role of organic osmolytes, growth regulators, and mineral nutrients,” in Physiological mechanisms and adaptation strategies in plants under changing environment, vol. 1. Eds. Ahmad, P., Wani, MR (New York, NY: Springer), 25-55. doi: 10.1007/978-1-4614-8591-9_2.
6. Sulaimon Basiru, Hopkins Pachalo Mwanza, and Mohamed Hijri. Analysis of Arbuscular Mycorrhizal Fungal Inoculant Benchmarks. Microorganisms 2021, 9, 81, https://doi.org/10.3390/microorganisms9010.
7. Rachel Backer1, J. Stefan Rokem2, Gayathri Ilangumaran1, et al. Plant Growth-Promoting Rhizobacteria: Context, Mechanisms of Action, and Roadmap to Commercialization of Biostimulants for Sustainable Agriculture. Front. Plant Sci., 23 Oct. 2018| https://doi.org/10.3389/fpls.2018.01473.
8. Okon, Y. and Labandera-Gonzalez, C. A. (1994) Agronomic Applications of Azospirillum: An Evaluation of 20 Years Worldwide Field Inoculation. Soil Biology and Biochemistry, 26, 1591-1601. http://dx.doi.org/10.1016/0038-0717(94)90311-5.
9. Glick, B. R. (1995) The Enhancement of Plant Growth by Free Living Bacteria. Canadian Journal of Microbiology, 41, 109-117. http://dx.doi.org/10.1139/m95-015.
10. F. Zaefarian, M. Rezvani, Soybean (Glycine max [L.] Merr.) Production Under Organic and Traditional Farming. Environmental Stresses in Soybean Production Soybean Production Volume 22016, Chapter 5. (Pages 103-129). https://doi.org/10.1016/B978-O-12-801535-3.00005-X.
11. Bonfante, P. & Genre, A. Mechanisms underlying beneficial plant-fungus interactions in mycorrhizal symbiosis. Nat. Commun. 1:48 doi: 10.1038/ncomms1046 (2010).
12. Wang, F.; Feng, G. Arbuscular mycorrhizal fungi interactions in the rhizosphere. In Rhizosphere Biology: Interaction between Microbes and Plants; Gupta, V. V. S. R., Sharma, A. K., Eds.; Springer: Singapore, 2021; pp. 217-235.
13. L. M. Egerton-Warburton, J. I. Querejeta, M. F. Allen, S. L. Finkelman. “Mycorrhizal Fungi,” in Encyclopedia of Soils in the Environment, 2005, vol. 2, Pages 533-542. https://doi.org/10.1016/B0-12-348530-4/00455-0.
14. M. J. ROSSI et al.: Production of Ectomycorrhizal Fungi by SSF and SmF, Food Technol. Biotechnol. 45 (3) 277-286 (2007).
15. M. A. Castellano, R. Molina: Mycorrhizae. In: The Biological Components: Nursery Pests and Mycorrhizae—The Container Tree Nursery Manual, T. D. Landis, R. W. Tinus, S. E. McDonald, J. P. Barnett (Eds.), US Department of Agriculture, Forest Service, Washington, DC, USA (1989) pp. 101-167.
16. M. B. Bogeat-Triboulot, F. Bartoli, J. Garbaye, R. Marmeisse, D. Tagu, Fungal ectomycorrhizal community and drought affect root hydraulic properties and soil adherence to roots of Pinus pinaster seedlings, Plant Soil, 267 (2004) 213-223.
17. J. M. Tisdall, S. E. Smith, P. Rengasamy, Aggregation of soil by fungal hyphae, Aust. J. Soil Res. 35 (1997) 55-60.
18. D. H Marx, A. B. Hatch, J. F. Mendicino, High soil fertility decreases sucrose content and susceptibility of loblolly pine roots to ectomycorrhizal infection by Pisolithus tinctorius, Can. J. Bot. 55 (1977) 1569-1574.
19. David Moore's World of Fungi: where mycology starts; http://www.davidmoore.org.uk/assets/mostly_mycology/diane_howarth/orchid.htm (accessed Dec. 17, 2021).
20. Frey-Klett, P.; Garbaye, J.; Tarkka, M. (2007-10-01). “The mycorrhiza helper bacteria revisited”. New Phytologist. 176 (1): 22-36. doi:10.1111/j.1469-8137.2007.02191.
21. Willey, Joanne M.; Sherwood, Linda M.; Woolverton, Christopher J. (2011). “Chapter 29: Microorganisms in Terrestrial Ecosystems”. Prescott's Microbiology. McGraw-Hill. pp. 703-706. ISBN 978-O-07-131367-4.
22. https://en.wikipedia.org/wiki/Ericoid_mycorrhiza (accessed Dec. 17, 2021).
23. Prabhukarthikeyan, S. R. et al., CHAPTER 3—“Bacillus rhizobacteria: A versatile biostimulant for sustainable agriculture.” New and Future Developments in Microbial Biotechnology and Bioengineering, Elsevier, 2022, Pages 33-44, ISBN 9780323851633, https://doi.org/10.1016/B978-O-323-85163-3.00009-0.
24. https://en.wikipedia.org/wiki/Mycorrhiza (accessed Dec. 17, 2021).

What is claimed is:

1. A method of growing a plant, comprising applying to the plant and/or growth medium for the plant (a) an effective amount of a composition comprising one or more mycorrhizal fungi and/or one or more mycorrhiza helper bacteria (MHB) and (b) an effective amount of a composition comprising one or more gibberellins, one or more auxins, salicylic acid, and/or one or more jasmonates.

2. The method of claim 1, wherein the applying of (a) and (b) occur concurrently or on the same day.

3. The method of claim 1, wherein the applying of (b) occurs 1 to 10 days after the applying of (a).

4. The method of any one of claims 1-3, further comprising applying the composition of (b) every 7 to 60 days after the applying of (a).

5. The method of any one of claims 1-4, wherein the MHB is one or more species in the genera Bacillus, Pseudomonas, and/or Streptomyces.

6. The method of any one of claims 1-4, wherein the PGPR is Pseudomonas putida, Azospirillum fluorescens, Azospirillum lipoferum, and/or one or more species in the genera Azotobacter, Klebsiella, Enterobacter, Alcaligenes, Arthrobacter, Burkholderia, Bacillus, Serratia, Allorhizobium, Azorhizobium, Bradyrhizobium, and/or Rhizobium.

7. The method of any one of claims 1-6, wherein prior to an optional dilution of the composition in (b):
the one or more gibberellins are at a concentration of 0.01 to 1 ng/ml; the one or more auxins are at a concentration of 0.001 to 1 ng/ml;
the salicylic acid is at a concentration of 0.1 to 1 ng/ml; and/or
the one or more jasmonates are at a concentration of 0.001 to 1 ng/ml.

8. The method of claim 1-6, wherein
the one or more gibberellins are at a concentration of 0.01 to 1 ng/ml; the one or more auxins are at a concentration of 0.001 to 1 ng/ml;
the salicylic acid is at a concentration of 0.1 to 1 ng/ml; and/or
the one or more jasmonates are at a concentration of 0.001 to 1 ng/ml; and
further comprising diluting the composition to 25:1 and 150:1 prior to applying the effective amount.

9. The method of any one of claims 1-8, wherein the composition in (b) is derived from algae, macroalgae, and/or microalgae.

10. The method of claim 9, wherein the microalgae comprise Chlorella, Spirulina, Nannochloropsis, and/or Scenedesmus or combinations thereof.

11. The method of any one of claims 1-10, wherein the one or more gibberellins comprise GA1, GA3, GA4, GA5, GA6, GA7, or combinations thereof.

12. The method of any one of claims 1-11, wherein the one or more auxins comprise Me-IAA, IAA-ALA, IAA-ASP, IBA, or combinations thereof.

13. The method of any one of claims 1-12, wherein the one or more jasmonates comprise jasmonic acid.

14. The method of any one of claims 1-13, wherein the composition in (b) further comprises cultured and enhanced sterilized water, optionally at 80% to 99.5% w/w.

15. The method of any one of claims 1-14, wherein the composition in (b) comprises biomass, ethylene, abscisic acid, brassinolides, or combinations thereof.

16. The method of any one of claims 1-14, wherein the composition in (b) does not comprise biomass, ethylene, abscisic acid, brassinolides, or combinations thereof.

17. The method of any one of claims 1-16, wherein the plant is from any of the following mycorrhizal host plant families: Actinidiaceae, Adoxaceae, Allaceae, Anacardiaceae, Apiaceae (Umbelliferae), Arecaceae, Asteraceae, Bromeliaceae, Cactaceae, Caesalpinioideae, Cannabaceae, Capparaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Rosaceae, Rutaceae, Sapindaceae, Saxifragaceae, Solanaceae, and/or Vitaceae.

18. The method of any one of claims 1-17, wherein the composition in (b) is derived from an alga, macroalgae, and/or microalgae.

19. The method of claim 18, wherein the microalgae comprise Chlorella, Spirulina, Nannochloropsis and/or Scenedesmus or any combination thereof.

20. The method of any one of claims 1-19, wherein the effective amount of the composition in (a) or (b) is applied to the one or more seeds, the soil, growth medium, and/or a cultivated area, and wherein the effective amount is 1 to 5 liters per acre.

21. The method of any one of claims 1-20, further comprising diluting with an aqueous solution the composition in (b) in a ratio of 50:1 and 300:1, respectively, and performing one or more additional applications of the effective amount of the composition.

22. The method of any one of claims 1-21, wherein the applying comprises injection, in-furrow, drip irrigation, fertigation, foliar application, seed placement, seed treatment, or combinations thereof.

23. The method of any one of claims 1-22, further comprising applying a nitrogen/phosphorus/potassium fertilizer.

24. A method of growing a plant, comprising
(a) providing an algae culture and air to an algae culture broth composition producing system, the system comprising:
a sterilizer;
an automatic carbon dioxide supply device to promote photosynthesis;
an at least partially or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and
a high-efficiency harvesting device using hollow fiber membranes;
(b) harvesting the composition;
(c) applying an effective amount of the composition to the plant and/or the growth medium; and
(d) apply an effective amount of a composition comprising one or more mycorrhizal fungi and/or one or more mycorrhiza helper bacteria (MHB).

25. The method of claim 24, wherein the composition in (b) is harvested by separating the composition from biomass of the algae culture using a harvesting device, optionally dehydrating the composition for storage and transport, and optionally diluting it prior to use.

26. The method of claim 24 or 25, wherein the algae culture comprises Chlorella, Spirulina, Nannochloropsis, and/or Scenedesmus, or any combination thereof.

27. The method of any one of claim 24-26, wherein the composition in (b) comprises one or more gibberellins, one or more auxins, salicylic acid, and/or one or more jasmonates.

28. The method of claim 27, wherein the one or more gibberellins comprise GA1, GA3, GA4, GA5, GA6, GA7 or combinations thereof.

29. The method of claim 27 or 28, wherein the one or more auxins comprise Me-IAA, IAA-ALA, IAA-ASP, IBA, or combinations thereof.

30. The method of any one of claims 27-29, wherein the one or more jasmonates comprise jasmonic acid.

31. The method of any one of claims 24-30, wherein the composition in (b) comprises biomass, ethylene, abscisic acid, brassinolides or combinations thereof.

32. The method of any one of claims 24-31, wherein the composition in (b) does not comprise biomass, ethylene, abscisic acid, brassinolides or combinations thereof.

33. A mixture comprising (a) a composition comprising one or more mycorrhizal fungi and/or one or more mycorrhiza helper bacteria (MHB) and (b) a composition comprising one or more gibberellins, one or more auxins, salicylic acid, and/or one or more jasmonates.

34. The use of one or more compositions for growing a plant, comprising:
(a) an effective amount of a composition comprising one or more mycorrhizal fungi and/or one or more mycorrhiza helper bacteria (MHB) and
(b) an effective amount of a composition one or more gibberellins; one or more auxins;
salicylic acid; and/or one or more jasmonates.
